(19) United States
(12) Patent Application Publication
(10) Pub. No.: US 2014/0037651 A1
(43) Pub. Date: Feb. 6, 2014

(54) DEPLETED ANTI-STAPHYLOCOCCAL ENTEROTOXINS POLYCLONAL ANTIBODIES, PREPARATION AND USES THEREOF

(71) Applicants: Gilles Prévost, Brumath (FR); Charline Barasino, Mulhouse (FR); Daniel Keller, Strasbourg (FR); Khaldoun Masoud, Afamia Latakia (SY)

(72) Inventors: Gilles Prévost, Brumath (FR); Charline Barasino, Mulhouse (FR); Daniel Keller, Strasbourg (FR); Khaldoun Masoud, Afamia Latakia (SY)

(73) Assignee: UNIVERSITÉ DE STRASBOURG, Strasbourg (FR)

(21) Appl. No.: 14/001,915

(22) PCT Filed: Feb. 28, 2012
(86) PCT No.: PCT/IB12/50909

§ 371 Date: Oct. 3, 2013

(30) Foreign Application Priority Data
Feb. 28, 2011 (EP) 11156131.2

(51) Int. Cl. G01N 33/569; C07K 16/12

(52) U.S. Cl. CPC G01N 33/56938; C07K 16/1271
USPC 424/165.1; 435/7.94; 530/389.5

(57) ABSTRACT

The present invention relates to the preparation of a set of depleted polyclonal antibodies, each depleted polyclonal antibody being raised against one specific staphylococcal enterotoxin, and its use for multiplex detection.

### TECHNICAL DOMAIN

The present invention relates to the multiplex titration of staphylococcal enterotoxins allowing simultaneous specific and quantitative detections with lower limits and with a higher magnitude of response. To this aim, the present invention uses affinity-purified and step-by-step immunoabsorbed or depleted polyclonal antibodies raised against staphylococcal enterotoxins.

The present invention finds an application in dairy products but also in human clinics, veterinary clinics, drugs control, meat and egg products.

In the specification below, references in square brackets ([ ]) refer to the list of references presented at the end of the text.

### STATE OF THE ART

An enterotoxin is a protein toxin released by a microorganism in the intestine. Enterotoxins are chromosomally encoded exotoxins that are produced and secreted from several bacterial organisms, for example Escherichia coli O157:H7, Clostridium perfringens, Vibrio cholerae, Staphylococcus aureus (also known as golden cluster seed, seed gold, or golden staph), Yersinia enterocolitica, Shigella dysenteriae. They are often heat-stable, and are of low molecular weight and water-soluble. Enterotoxins are frequently cytotoxic and kill cells by altering the apical membrane permeability of the mucosal (epithelial) cells of the intestinal wall. They are mostly pore-forming toxins (mostly chloride pores), secreted by bacteria, that assemble to form pores in cell membranes. This causes the cells to die.

Staphylococcal enterotoxins (SEs) are basic proteins highly toxic produced by certain Staphylococcus strains in a variety of environments, including food substrates. SEs are exoproteins with a molecular weight between 22 and 29 kDa showing neurotoxic properties in humans. In addition, SEs are powerful superantigens that stimulate non-specific T-cell proliferation, and share close phylogenetic relationships, with similar structures and activities. To date, 23 different SEs have been identified. Types of toxins SEA, SEB, SEC, SED and SEE represent “conventional” toxins because well-characterized and identified for many years, and which involvement in cases of food poisoning has been shown by many authors [Jones and Kahn, J. Bacteriol., 166: 29-33, 1986; Betley and Mekalanos, J. Bacteriol., 170(1): 34-41, 1988; Couch and al., J. Bacteriol., 170: 2954-2960, 1988; Bayles and Iandolo, J. Bacteriol., 171: 4799-4806, 1989; Dingues and al., Clin. Microbiol. Rev., 13: 16-34, 2000] [1-5]. Serotype C was subdivided in groups (SEC1, SEC2, SEC3, SEC_{bovine}, SECₒᵥᵢₙₑ, SEC_{goat} and SEC_{canine}) classified according to differences of superantigen activity and depending on the host to which they are associated [Bergdoll and al., J. Bacteriol., 90(5): 1481-1485, 1965; Marr and al., Infect. Immun., 61: 4254-4262, 1993] [6, 7]. A staphylococcal enterotoxin SEF was described in 1981 [Bergdoll, Lancet, 1: 1017-1021, 1981] [8], but was renamed a few years later TSST1 given its lack of emetic activity unlike other conventional enterotoxins [Blomster-Hautamaa and al., J. Biol. Chem., 261: 15783-15786, 1986] [9]. Since the mid-1990 s, from the genome analysis of S. aureus, several genes with strong sequence homology with genes of conventional enterotoxins have been identified. These genes encode proteins with structural properties similar to those of enterotoxins whose emetic activity has rarely been demonstrated. In 2004, a new nomenclature for the superantigens expressed by S. aureus has been proposed for the designation of these new enterotoxins [Lina and al., J. Infect. Dis., 189(12): 2334-2336, 2004] [10]. Accordingly, only the toxins inducing emetic activity after oral administration in animals have been designated staphylococcal enterotoxins (SEs), namely types of toxins SEA, SEB, SEC, SED, SEE, SEG, SEH and SEI. Other toxins, for which no emetic activity has been demonstrated in vivo, have been called “staphylococcal enterotoxin-like” to indicate that their role in food poisoning was not confirmed [Ren et al., J. Exp. Med., 180(5): 1675-1683, 1994; Jarraud and al., J. Immunol., 166(1): 669-677, 2001; Orwin and al., Infect. Immun., 69(1): 360-366, 2001; Letertre and al., Mol. Cell Probes, 17: 227-235, 2003; Omoe and al., J. Clin. Microbiol., 40: 857-862, 2002; Su and Wong, J. Food Prot., 59(3): 327-330, 1996; Munson and al., Infect. Immun., 66: 3337-3348, 1998] [11-17]. In 2008, two new enterotoxins SES and SET were cloned and purified which show an emetic activity in animals, as newly confirmed for the enterotoxin SER [Ono and al., Infect. Immun., 76(11): 4999-5005, 2008] [18].

The most sensitive methods to detect toxins are the polymerase chain reaction (PCR) and enzyme-linked immunoabsorbent assay (ELISA). The PCR-related methods are more suitable for detection of organisms, such as bacteria and viruses, from which nucleic acids can be extracted for specific amplification. The ELISA-based methods are more robust for detection of toxins, which are often proteins in nature, using specific polyclonal or monoclonal antibodies when available. In comparison with the PCR method, the ELISA-based detection is less sensitive to the matrix effect and presumably gives fewer false-positive results, and ELISA can be addressed to thermostable proteins while bacteria may disappear in slightly heated foods.

All the staphylococcal toxins share similarities in amino acid sequence homology ranging from 15.5% (between SEB and SEK) to 81% (between SEA and SEE) which can lead to lack of specificity of tools used for their detection. Moreover up to date, the titration of staphylococcal enterotoxins is achieved antigen by antigen (which is extremely time consuming) and it is proposed to a limited number (4 or 5) of staphylococcal enterotoxins involved in food-born intoxination issued from dairy products. Commercial kits are qualitative but when quantitative, they generally lack specificity (which does not allow to easily conduct epidemiological links) and/or sensitivity (which puts the tests over the limits of human toxicity threshold of staphylococcal enterotoxins). Furthermore no commercial kit has been able to fulfil national and international standards (e.g. AFNOR in France).

Therefore, there is a real need to identify new methods for detection and specific titration of staphylococcal enterotoxins, that overcomes the shortcomings, disadvantages and obstacles of prior art, and thereby improving the management of staphylococcal enterotoxins contaminations, notably those induced by enterotoxins from Staphylococcus aureus, while reducing costs and time consumption.

### DESCRIPTION OF THE INVENTION

The Inventors have now developed unexpectedly a multiplex assay that allows simultaneously the specific titration of at least staphylococcal enterotoxins A, B, C, D, E, G, H and I. Any of the cited antigens are expressed in a recombinant form that remains functional and does not harbour significant difference in their epitopes. These antigens are used for immunizations, and serve as controls in the test assays. The titration was based on the Luminex® technology and uses rabbit polyclonal antibodies that were affinity-purified, and secondarily step-by-step immunoabsorbed or depleted against cross-reacting enterotoxins to reach a strict specificity which preserves affinity and sensitivity of a bulk of specific polyclonal antibodies only for the corresponding antigen. The multiplex titration finally allows simultaneous specific and quantitative detections with lower limits close to 50-80 pg/g of initial products, e.g. culture supernatants, dairy products, human serum and with at least a more than two logarithmic magnitude of response. Such a simultaneous titration offers homogeneity and possibilities to detect toxin concentrations that would remain below the human toxicity threshold. Such a simultaneous titration conjoined to a low consummation of the developed products brings decreased cost compared to those actually observed for these assays and reduces time for results.

The present invention, therefore, relates to an immunological composition comprising a mixture of at least two depleted polyclonal antibodies each depleted polyclonal antibody being raised against one specific staphylococcal enterotoxin, and a pharmaceutically acceptable carrier. Preferably, it relates to an immunological composition comprising a mixture of at least three, four, five, six, seven or eight depleted polyclonal antibodies each depleted polyclonal antibody being raised or directed against one specific staphylococcal enterotoxin, and a pharmaceutically acceptable carrier. According to the present invention, it may be in particular depleted polyclonal antibodies each polyclonal antibody being raised against one specific staphylococcal enterotoxin chosen from the group consisting of the staphylococcal enterotoxins A, B, C, D, E, G, H, and I.

“Depleted polyclonal antibodies” within the meaning of the present invention means a set of polyclonal antibodies, each polyclonal antibody being raised and/or directed against a given staphylococcal enterotoxin (immunogen), different from each other, that is affinity-purified and secondarily step-by-step immunoabsorbed (or depleted) against cross-reacting enterotoxin(s) that are different from the staphylococcal enterotoxin used as immunogen, to reach a strict specificity which preserves affinity and sensitivity of a bulk of specific polyclonal antibodies only for the corresponding given staphylococcal enterotoxin (immunogen). Said step-by-step immunoabsorbed or depleted polyclonal antibodies are not conjugated to a support as conventional immunoabsorbed antibodies at the end of steps of purification and step-by-step immunoabsorbtion/depletion, but can be subsequently linked to a support if necessary.

“Pharmaceutically acceptable carrier” within the meaning of the present invention means any and all solvents, disintegrating agents, binders, excipients, lubricants, absorption delaying agents and the like.

“Staphylococcal enterotoxins A, B, C, D, E, G, H and I” within the meaning of the present invention means the staphylococcal enterotoxins (SEs), namely types of toxins SEA, SEB, SEC, SED, SEE, SEG, SEH and SEI, produced by Staphylococcus aureus.

The present invention also relates to a method for multiplex detection of staphylococcal enterotoxins, comprising:

a) contacting a sample with an immunological composition of the invention;

b) detecting potential immunological complexes formed.

“A sample” within the meaning of the present invention means any culture media, biological samples (e.g. human clinical samples) or food extracts that are susceptible of being contaminated by staphylococcal enterotoxins. For example, it includes culture supernatants, human serum, dairy/egg/meat/prepared meals/sea foods products/spices/drugs and surface of medical devices, and any extracts or protein preparation issued from the cited stuffs.

“Immunological complexes” within the meaning of the present invention means the complexes formed between the depleted polyclonal antibody and its immunogen (namely the given staphylococcal enterotoxin used for immunization).

The detection of said immunological complexes can be performed by any means known in the art. For example, it includes protein mass—based technologies (e.g. protein CHIPS assisted by SELDI-TOF or MALDI-TOF, fluorescence-based technologies (e.g. Luminex technology), surface plasmon resonnance-based technologies (biosensors).

According to a particular embodiment of the present invention, said method for multiplex detection can also include a step c) wherein the detection results obtained in step b) are compared to a negative and/or positive control.

“Positive and/or negative control” within the meaning of the present invention means a control sample comprising or not at least one staphylococcal enterotoxin, respectively. This control allows to compare the detection results obtained in step b) of the method of the invention and to detect a false positive or false negative, if any.

According to a particular embodiment of the present invention, said method of multiplex detection can also include a step c_{bis}) wherein the detection results obtained in step b) are compared to a standard of measurement; allowing a qualitative and/or quantitative analysis of staphylococcal enterotoxins.

“Standard of measurement” within the meaning of the present invention means one or more analysis carried out on solutions manufactured for analytical purposes according to the invention and comprising a known staphylococcal enterotoxin(s) content. From the analysis results, it is thus possible to determine the presence of staphylococcal enterotoxin(s) in the sample as well as its concentration by comparison, for example using a standard curve made from standards or measurement.

According to the present invention, steps c) and c_{bis}) can be both implemented, before or after steps a) and b) of the method of the invention. According to the present invention, steps c) and c_{bis}) can be implemented simultaneously or sequentially, including to steps a) and b), for example on a multi-well plate for performing several analysis simultaneously or sequentially, including measures of standards.

The present invention also relates to the use of an immunological composition of the invention, as a diagnostic tool of a staphylococcal enterotoxin(s) contamination.

Such a diagnostic tool allows a qualitative and/or quantitative diagnosis of a staphylococcal enterotoxin(s) contamination.

The present invention also relates to a kit for the multiplex detection of staphylococcal enterotoxins, comprising an immunological composition of the invention and means for the detection of immunological complexes.

“Means for the detection of immunological complexes” within the meaning of the present invention means any means well known in the art. For example, it includes Cy5, phycoerythrin or any other fluorescent dyes, fluorescent metabolites issued from enzymes activity, Matrix assisted laser desorption ionization procedures, Fluorescent resonance energy transfer, surface plasmon resonance.

The present invention also relates to a method for the preparation of a depleted polyclonal antibody raised and/or directed against one specific staphylococcal enterotoxin, comprising:

a) providing an anti-enterotoxin polyclonal antibody previously obtained from the immunization of a non-human animal with a given staphylococcal enterotoxin;

b) purification of said anti-enterotoxin polyclonal antibody using at least two successive immunoabsorption (or depletion) steps against immunization-unrelated staphylococcal enterotoxins and which order is chosen to abolish cross-reactions with said immunization-unrelated staphylococcal enterotoxins from the strongest cross-reaction to the weaker cross-reaction.

“Immunization-unrelated staphylococcal enterotoxins” within the meaning of the present invention means given staphylococcal enterotoxin(s) Y (SEY) that differ(s) from a given staphylococcal enterotoxin X (SEX), used as immunogen for obtaining a polyclonal antibody raised against said given staphylococcal enterotoxin X (SEX), but that is responsible for a cross reaction with said anti-SEX polyclonal antibody.

For example, step b) is carried out by purification/depletion of an affinity purified anti-SE polyclonal antibody on an immunoabsorption column with a first immunization-unrelated staphylococcal enterotoxin immobilized thereon responsible for a cross reaction. If a cross reaction remains, a new purification/depletion is carried out on an immunoabsorption column with the same or another immunization-unrelated staphylococcal enterotoxin responsible for the cross reaction. These purification steps are repeated until obtaining monospecificity of depleted polyclonal antibody with respect to its immunogen. The order of successive columns can be easily determined by one skilled in the art according to the extent of each cross reaction detected, namely from the stronger cross reactivity to the weaker cross-reactivity. Preferably, said method for the preparation of a depleted anti-SE polyclonal antibody can also include washing steps between each purification step.

According to a particular embodiment of the present invention, said method for the preparation of a depleted anti-SE polyclonal antibody can also include a step c) wherein the specificity of said depleted polyclonal antibody is monitored by any means well known in the art. For example, the specificity of the depleted anti-SE polyclonal antibody can be controlled by ELISA, DOT-BLOT against its immunogen and/or one or more parent staphylococcal enterotoxin(s) to detect the presence of remaining cross reaction, if any. Preferably, one or more analysis is(are) carried out on solutions manufactured for analytical purposes according to the invention and comprising the known immunogen or an immunization-unrelated staphylococcal enterotoxin content. From the analysis results, it is thus possible to determine the presence of any cross reaction as well as its extent by comparison, for example using a standard curve made from standards of measurement.

According to the present invention, step c) can be implemented between each depletion step and/or at the end of step b). Preferably step c) is implemented between each depletion step to detect remaining cross reaction(s), determine the extent of each cross reaction detected, and thus determine the necessary next depletion step(s) to achieve monospecificity.

According to a particular embodiment of the present invention, the method for the preparation of a depleted polyclonal antibody against SEA comprises a step b) wherein 5 successive depletion steps against the immunization-unrelated staphylococcal enterotoxin E, E, I, B, then D are implemented.

According to a particular embodiment of the present invention, the method for the preparation of an immunoabsorbed polyclonal antibody against SEB comprises a step b) wherein 3 successive depletion steps against the immunization-unrelated staphylococcal enterotoxin C1, C1, then G are implemented.

According to a particular embodiment of the present invention, the method for the preparation of a depleted polyclonal antibody against SEC comprises a step b) wherein 3 successive depletion steps against immunization-unrelated staphylococcal enterotoxin B, B, then G are performed.

According to a particular embodiment of the present invention, the method for the preparation of a depleted polyclonal antibody against SED comprises a step b) wherein 3 successive depletion steps against the immunization-unrelated staphylococcal enterotoxin A, A, then E are performed.

According to a particular embodiment of the present invention, the method for the preparation of a depleted polyclonal antibody against SEE comprises a step b) wherein 4 successive depletion steps against the immunization-unrelated staphylococcal enterotoxin A, A, I, then C1 are performed.

According to a particular embodiment of the present invention, the method for the preparation of a depleted polyclonal antibody against SEG comprises a step b) wherein 4 successive depletion steps against the immunization-unrelated staphylococcal enterotoxin A, B, I, then C1 are performed.

According to a particular embodiment of the present invention, the method for the preparation of a depleted polyclonal antibody against SEH comprises a step b) wherein 2 successive depletion steps against the immunization-unrelated staphylococcal enterotoxin B, then D.

According to a particular embodiment of the present invention, the method for the preparation of a depleted polyclonal antibody against SEI comprises a step b) wherein 5 successive depletion steps against immunization-unrelated staphylococcal enterotoxin E, C1, G, B, then A are performed.

The present invention also relates to a depleted anti-SE polyclonal antibody raised against a staphylococcal enterotoxin obtained by a method for the preparation of a depleted anti-enterotoxin polyclonal antibody of the invention. Such depleted anti-enterotoxin polyclonal antibodies differ from those of the art by a different population of antibodies, affinity and specificity to the corresponding antigen.

For example depleted anti-SE polyclonal antibodies of the invention are available free in solutions or adsorbed on a support, for example a bead (microsphere or nanoparticle), but can be adsorbed also onto a protein microarray, a functionalized multiwell plate, etc. . . . Preferably, such depleted polyclonal antibodies are adsorbed on beads, more preferably on beads of an unique color-code according to the strict specificity of the depleted anti-SE polyclonal antibody of the invention adsorbed thereon.

### BRIEF DESCRIPTION OF THE FIGURES

FIG. 1 represents the pGEX-6P-1 expression vector containing a resistance gene to ampicillin and two restriction sites for BamH1 (loc.945) and EcoR1 (loc. 954).

FIG. 2 represents the amino acids sequences of staphylococcal enterotoxinsSEA-I.

FIG. 3 represents the synthesis of the calibration curves for plates 100 to 104

FIG. 4 represents the titration curves of staphylococcal enterotoxins SEA-I.

FIG. 5 represents the LOD and LOQ for plates 100 to 104+/− standard deviation.

FIG. 6 represents the linearity of standard for plates 100 to 104.

FIG. 7 represents SEs titration on Munster after caseins precipitation and delipidation

FIG. 8 represents SEs titration on concentrated matrix after caseins precipitation and delipidation.

### EXAMPLES

### Example 1

### Origins of the 8 Staphylococcal Enterotoxins

Sources of the Basic Staphylococcus aureus Strains Used for Cloning

The genes fragments corresponding to the 8 Staphylococcal enterotoxins (SEs) secreted sequences were obtained from strict amplification (Phusion™ High Fidelity DNA Polymerase) of genes from strains listed in the table 1 below.

TABLE 1

| Enterotoxin | Strain (genotype) | Source |
|---|---|---|
| SEA | FRI722 (sea) | DeBuyser |
| SEB | S6 | DeBuyser |
| SEC | FRI137 (sec1, seg, seh, sei) | DeBuyser/AFSSA |
| SED | FRI361 (sec, sed, seg, sei)/ | DeBuyser |
| | FRI1157m | |
| SEE | FRI 326 | DeBuyser |
| SEG | FRI137 (sec, seg, seh, sei) | AFSSA |
| SEH | FRI137 (sec, seg, seh, sei) | AFSSA |
| SEI | FRI137 (sec, seg, seh, sei) | AFSSA |

Cloned genes have been checked by nucleotide sequencing. Oligonucleotides adjustable to the plasmid pGEX-6P-1 were used for an insertion either in the EcoR1 cut site (protein+8 amino acids), or in the BamH1 cut site (protein+5 amino acids). Antigens are over-expressed from recombinant clones pGEX-6P-1 (SEA+5, SEB+5, SEC1+8, SEE+5, SEG+8, SEH+5 and SEI+5) transforming BL21 as Glutathion S-Transferase (GST) fusion proteins, then purified by glutathion affinity (Sepharose4B GSH) followed by a cation exchange chromatography (Mono S), adapted to each SE. All SEs have purity over 90-95% in SDS-PAGE. SEA+5, SEB+5, SEC1+8 are recognized by the kit Oxoid SET RPLA and SEE+5 by Oxoid anti-SEA from the same kit.

### Bacterial Strains

Escherichia. coli XL1-Blue

Genotype: recA1 endA1 gyrA96 thi-1 hsdR17 supE44 relA1 lac [F′ proAB lacl^{q}ZΔM15 Tn 10 (Tet^{r})]

Escherichia coli BL21

Genotype: E. coli B F— dcm ompT hsdS (r_{B}- m_{B}-) gal
Plasmid Vector (pGEX-6P-1)

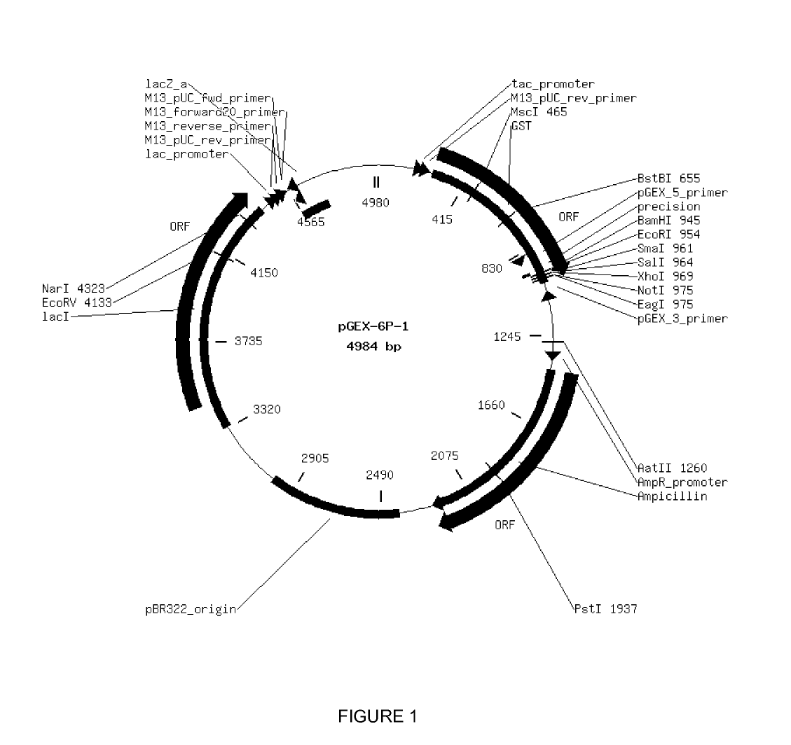

It is a 4984 pb vector (FIG. 1) containing an ampicillin resistance gene, a gene coding to the Glutathion-S-Transferase (GST) and a multiple cloning region located in 3′ of that gene.

Cloned sequences in multiple restriction/insertion sites of pGEX-6P-1 vector can be expressed as GST fusion proteins located at its N-terminal extremity [Smith and Johnson, Gene, 67(1): 31-40, 1988] [19]. Expression is under the control of the ptac promoter, upstream the GST coding gene, which is induced by the isopropyl β-D thiogalactopyranoside (IPTG). The fusion protein expressed in the pGEX-6P-1™ can easily be purified by affinity chromatography using a Glutathion Sepharose 4B™ column (GE Healthcare, Orsay). The fusion protein contains a specific proteolysis site using PreScission™ Protease (GE Healthcare, Orsay), and is located between the GST and its fusion partner.

### Recombined Vectors

Recombined vectors used are made in the first part of work by the cloning method (table 2).

TABLE 2

| Plasmids | Relatives characteristics (Origin) |
|---|---|
| pGEX-6P-1 | Ap^{r}, GST expression vector (Pharmacia) |
| pPAX1 | Ap^{r}, pGEX-6P-1 carrying sea (Gravet et al.) |
| pKBX1 | Ap^{r}, pGEX-6P-1 carrying seb (That work) |
| pKCX1 | Ap^{r}, pGEX-6P-1 carrying sec1 (That work) |
| pPDX1 | Ap^{r}, pGEX-6P-1 carrying sed (That work) |
| pKEX1 | Ap^{r}, pGEX-6P-1 carrying see (That work) |
| pKGX1 | Ap^{r}, pGEX-6P-1 carrying seg (That work) |
| pKHX1 | Ap^{r}, pGEX-6P-1 carrying seh (That work) |
| pKIX1 | Ap^{r}, pGEX-6P-1 carrying sei (That work) |

### DNA Primers Used for Genetic Amplification (PCR)

Oligonucleotides used for the genetic amplification of the genes coding the staphylococcal enterotoxins are shown in the table 3 below.

TABLE 3

| | Accession | | | | Tm |
|---|---|---|---|---|---|
| Gene | N^{o} | Position | Primers | Primers sequence 5′→3′ | (° C.) |
| seb | M11118 | 325-1041 | BamH1-setBf | AGTAAGGATCCGAGAGAGTCAAC | 75 |
| | | | | CAGATCCTAAACCA | |
| | | | | (SEQ ID NO: 1) | |
| | | | EcoR1-setBr | AAGTATAGAATTCACTAACACTTC | 64 |
| | | | | ATATTTACTTTCTAAAAT | |
| | | | | (SEQ ID NO: 2) | |
| sec1 | XO5815 | 199-915 | EcoR1-setC1f | AAGTATAGAATTCGAGAGCCAAC | 79 |
| | | | | CAGACCCTACGCCAGA | |
| | | | | (SEQ ID NO: 3) | |
| | | | EcoR1-setC1r | AAGTATAGAATTCTACTTTCACTT | 67 |
| | | | | TTTATATCAAAATGG | |
| | | | | (SEQ ID NO: 4) | |
| sed | M28521 | 445-1065 | EcoR1-setDf | AAGTATAGAATTCAAACATTCTTAT | 64 |
| | | | | GCAGATAAAAATCCAAT | |
| | | | | (SEQ ID NO: 5) | |
| | | | EcoR1-setDr | AAGTATAGAATTCTTGAAATGGCT | 63 |
| | | | | TTAGTGTCTGATGTT | |
| | | | | (SEQ ID NO: 6) | |
| see | M21319 | 82-771 | BamH1-setEf | AGTAAGGATCCAGCGAAGAAATA | 65 |
| | | | | AATGAAAAAGATTT | |
| | | | | (SEQ ID NO: 7) | |
| | | | EcoR1-setEr | AAGTATAGAATTCAGTTGTGTATA | 57 |
| | | | | AATACAAATCAATAT | |
| | | | | (SEQ ID NO: 8) | |
| seg | AF064773 | 229-927 | EcoR1-seGf | AGTAAGGATCCCAACCCGATCCT | 68 |
| | | | | AAATTAGACGAAC | |
| | | | | (SEQ ID NO: 9) | |
| | | | EcoR1-seGr | AAGTATGAATTCTCAGTGAGTATT | 62 |
| | | | | AAGAAATACTTCCATTTT | |
| | | | | (SEQ ID NO: 10) | |
| seh | U11702 | 280-930 | BamH1-setHf | AGTAAGGATCCGAAGATTTACAC | 64 |
| | | | | GATAAAAGTGAGTT | |
| | | | | (SEQ ID NO: 11) | |
| | | | EcoR1-setHr | AAGTATAGAATTCTGATCTAGAAA | 58 |
| | | | | TTTTCATTGATTATA | |
| | | | | (SEQ ID NO: 12) | |
| sei | AF064774 | 226-879 | BamH1-setIf | AGTAAGGATCCCAAGGTGATATT | 65 |
| | | | | GGTGTAGGTAACTT | |
| | | | | (SEQ ID NO: 13) | |
| | | | EcoR1-setIr | AAGTATAGAATTCGTTACTATCTA | 57 |
| | | | | CATATGATATTTCG | |
| | | | | (SEQ ID NO: 14) | |

### Media

2×TY (Trypcase-Yeast extract) medium: 1.6% (p/v) bio-trypcase (BioMérieux), 1% (p/v) yeast extract (Bio-Rad), 0.5% (p/v) NaCl pH 7.4. The agar medium contains 1.5% (p/v).
2×TYA medium: 2×TY medium containing 100 μg/mL of ampicilline.
M9 medium: 15 g/L Na₂HPO₄-12H₂O, 3 g/L KH₂PO₄, 1 g/L NH₄Cl, 0.5 g/L NaCl, 15 g/L agar. Following the autoclave add: 1 mL 0.1 M CaCl₂, 1 mL 1M MgSO₄, 1 mL 1M Thiamine-HCl, 2 mL 20% (p/v) Glucose
Strains freezing medium: 90% (v/v) Brain Heart Infusion (Difco), 10% (v/v) glycerol (87%).

### Reagents and Buffers

TEB 10×: 0.89 M Tris-base, 0.89 M boric acid, 25 mM EDTA-Na₂ (Titriplex III), pH 8.3.

TE: 10 mM Tris-HCl, 1 mM EDTA-Na2, pH 8.0.

TEG: 25 mM Tris-HCl, 50 mM Glucose, 10 mM EDTA; pH 8.0.

Alkaline Lysis Buffer(ALB): 0.2 M NaOH, 1% (m/v) SDS.

Plasmid DNA preparation reagents (Plasmid-combi-kit, Qiagen):

Buffer P1: 50 mM Tris-HCl, 10 mM EDTA-Na₂ pH 8.0; 100 μg/mL RNase A.

Buffer P2: 200 mM NaOH, 1% (m/v) SDS.

Buffer P3: 3.0 M Potassium acetate, pH 5.5.

Buffer QC: 50 mM MOPS, 1 M NaCl, pH 7.0, 15% (v/v) ethanol.

Buffer QBT: 50 mM MOPS, 750 mM NaCl, pH 7.0, 15% (v/v) ethanol, 0.15% (v/v) Triton X100.

Buffer QF: 50 mM Tris-HCl, 1.25 M NaCl, pH 8.5, 15% (v/v) ethanol.

×5 DNA denaturating solution: 25 mM EDTA-Na₂ pH 8.0, 20% (m/v) glycerol, 0.5% (m/v) lauroyl sarcosine, 0.1% (m/v) Bromophenol blue. This solution leads to DNA denaturation and can be used as a migration control in agarose gel electrophoresis.

SDS-PAGE loading buffer: 0.2 M Tricine, 0.2 M Tris-base, 0.55% (m/v) SDS, pH 8.0.

Coomassie blue coloration stock solution: 0.2 g Coomassie blue G250 R (GE Healthcare), 60% (v/v) methanol, QSP 200 mL with H₂O.

Coomassie Blue coloration finale solution: 50% (v/v) of filtered stock solution, 10% (v/v) acetic acid, 30% (v/v) methanol.

PBS: 2.7 mM KCl, 10 mM, Na₂HPO₄, 1.8 mM KH₂PO₄, 140 mM NaCl, pH 7.4.

Lysis Buffer A: 20 mM HEPES, NaCl 150 mM, EDTA 1 mM pH 7.2.

Buffer B: 100 mM Acetic acid, 500 mM NaCl, pH 4.0.

Elution buffer C: 50 mM Tris-hydroxyamino methyl-HCl, 500 mM, NaCl, 30 mM GSH, pH 8.0.

### Preparation of Staphylococcus aureus Total DNA

From a 5 mL preculture of the corresponding strain of S. aureus, inoculate 2×10 mL of 2×TY medium at 37° C. overnight. Spin 10 min at 5000×g. Wash the pellet with water. Resuspend in 7 mL of lysis buffer [50 mM Tris; 25% Sucrose; 2 mg/mL lysozyme; 0.05 mg/mL Lysostaphine; pH 7.5] and 100 μL of 2 μg/mL of boiled RNase A. Incubate 45 min at 37° C. Spin 10 min at 5000×g. Resuspend the pellet in 3852 μL of water, 300 μL of EDTA, NA₂ 250 mM pH 8.0 and 300 μL of SDS 10% (v/v). Agitate vigorously. Add 10 mL of phenol, equilibrated at pH 8.0. Agitate vigorously. Spin 5 min at 5000×g. The upper aqueous phase undergoes a second extraction with 10 mL of phenol/chloroform (1/1). Eventually repeat that step. Precipitate the DNA with 3 volumes of absolute ethanol. Save the precipitate and move it into a new tube. Wash with 70% ethanol (v/v) and spin 5 min at 5000×g. Repeat that step. Dry the DNA pellet. Dissolve in 300 μL of TE buffer. Store at −20° C.

DNA amplification by Polymerase Chain Reaction (PCR)

Assemble the reaction in ice, in that order: 35.5 μL of MQ water, 10 μL of PCR×5 buffer (which contains 7.5 mM MgCl2), 1 μL of 0.2 mM dNTP, 1 μL of each corresponding 25 μM primer solutions (forward and reverse), 1 μL of the corresponding S. aureus total DNA (about 100 μg) and 0.5 μL of the DNA polymerase (Phusion High Fidelity DNA polymerase). PCR cycles for the amplification of the SEs genes are shown in the table 4 below.

TABLE 4

| Phase | Length | Temperature | N^{o} of cycles |
|---|---|---|---|
| Initial denaturation | 40 s | 94° C. | 1 |
| Initial denaturation | 20 s | 94° C. | |
| Hybridization | 30 s | 52° C. | 35 |
| Elongation | 2 min | 72° C. | |
| Final elongation | 3 min | 72° C. | 1 |

Control of PCR Product on Agarose Gel (1% w/v)

The agarose is dissoluted in TBE buffer×1 (89 mM Tris-borate, 2 mM EDTA, pH 8.0) by heating. Ethidium Bromide is included in the gel matrix at 0.5 μg/mL to enable fluorescent visualisation of the DNA fragment under UV light (254 nm to 300 nm). The migration occurs at 110V during about 1 h.

Electroelution of the PCR product in low melting point agarose gel 1.8% at 100V

Make a phenol/chloroform extraction, then one ether extraction. Add 3 μL of 5M NaCl and precipitate the DNA with ice-cold absolute ethanol. Wash the pellet with 70% ethanol (v/v) and let dry. Resuspend the DNA in TE and store at −20° C.

### Ligation of the Insert in the Plasmid

Restriction Hydrolysis

Assemble in a Eppendorf tube: 15 μL of the restriction enzyme buffer×10, 110 μL of water, 20 μL (10 μg) of the plasmid or insert from PCR and 5 μL of the corresponding enzyme restriction. Incubate 2 h at 37° C. Add 6 μL of 250 mM EDTA pH8.0. Make a phenol extraction, keep the aqueous phase (upper phase). Make three ether extractions, keep the aqueous phase (lower phase). Add 3 μL of 5M NaCl and 400 μL of ice-cold absolute ethanol. Spin 5 min at 7 000×g at 4° C., and discard the supernatant. Wash the DNA pellet with 70% ethanol (v/v), spin and discard the supernatant. Dry the DNA pellet. Continue to the following step: plasmid dephosphorylation.

Plasmid Dephosphorylation

To the dried DNA pellet, add 134 μL of MQ water, 15 μL of the buffer×10 and 1 μL of the CAIP enzyme. Incubate 1 h at 37° C. Add the same volumes and re-incubate 1 h at 37° C. Add 12 μL of 250 mM EDTA pH 8, vortex. Add 250 μL of phenol. Vortex and incubate 10 min at 65° C. Vortex and re-incubate 10 min at 65° C. Discard the phenol. Make a phenol/chloroform extraction, then four ether extractions. Add 3 μL of 5M NaCl and precipitate the DNA with ice-cold absolute ethanol. Wash the pellet with 70% ethanol (v/v) and let dry. Resuspend the DNA in 125 μL of TE and store at −20° C.

Ligation of the Plasmid and the DNA Fragment

In a final volume of 15 μL, assemble ligase buffer; a molar ratio insert/vector of 3 to 5 and 0.2 U of T4 DNA ligase. Incubate 16 h at 14° C.
Transformation of Competent Bacteria with the Ligation Product

Preparation of Competent Cells

From 3 mL of 2×TY preculture, incubate a 100 mL culture of E. coli XL1 Blue at 37° C. under 200 rpm shaking in the same medium. When OD₆₀₀ₙₘ reach 0.5 to 0.7, spin 10 min at 2500×g. Resuspend the pellet in 10 mL of 50 mM CaCl₂. Leave the bacteria 30 min at 4° C., then spin 10 min at 2500×g at 4° C. Resuspend the pellet in 2.5 mL of 50 mM CaCl₂.

Transformation of Bacteria by the Thermal Shock Method

Add 4 μL of ligation product to 150 μL of competent E. coli XL1 Blue cells. Incubate 40 min at 4° C., then a thermal shock of 2 min at 42° C. After 2 min of chilling at 4° C., spread out the preparation on TYA plate and incubate at 37° C. overnight.

### Control of Ligation

Mini Preparation of Plasmid DNA from E. Coli

Inoculate a 2×TYA medium (3 mL) with an isolated colony and grow overnight at 37° C. Pellet cells at 12 000 xg for 30 sec. and discard the supernatant. Add 100 μL of TEG buffer [25 mM Tris, 50 mM glucose, 10 mM EDTA, pH 8.0] and resuspend cells. Add 200 μL of Alkaline Lysis Buffer [0.2N NaOH, 1% SDS], mix by inverting 5-6 times. Add 150 μL of 3M Potassium Acetate (pH 5.5). Spin 10 min at 12 000×g. Transfer 400 μL of supernatant to a new tube containing 1 mL of 24:1 Chloroform:Isoamyl Alcohol (CHCl₃:IAA) and mix by inverting. Spin at 12 000×g for 10 min. Transfer aqueous phase to 800 μL of ice-cold absolute ethanol. Precipitate at −20° C. for at least 30 min. Spin at 7 000×g for 10 min. Discard the supernatant and wash the pellet with 1 mL 70% ethanol (v/v), then 1 mL 95% ethanol (v/v). Discard ethanol and dry the pellet (air-dry). Dissolve in 40 μL of TE containing 20 μg/mL of RNase A (Ribonuclease A SERVA®). Store at −20° C.

Control of Ligation Product by Agarose Gel Electrophoresis

DNA minipreps are digested by the restriction enzyme corresponding to the insertion sites. The restriction product is then analyzed by agarose gel electrophoresis.

Maxi preparation of DNA by chromatography (Plasmid Combi kit, Qiagen) Inoculate 100 mL of 2×TYA medium with transformed bacteria and grow overnight at 37° C. Spin 10 min at 5000×g. Resuspend the cells in 6 mL of P1 buffer. Transfer the solutions into ultracentrifugation tubes. Add 6 mL of P2 buffer in order to lyse the bacteria. Incubate 5 min and add 6 mL of P3 buffer (SDS is precipited and the mixture is becoming viscous). Leave the medium 5 min at room temperature then spin 40 min at 25 000×g at 4° C. in angular rotor (Ti 60-Beckman). Supernatant is then used for the chromatography.

Equilibrate the anions exchange column (DEAE-cellulose groups) with 10 mL of QBT buffer. Apply the supernatant to the column and then wash with 2×10 mL of QC buffer. Eluate the DNA with 7 mL of QF buffer. Add 20 mL of ethanol and spin 10 min at 5000×g at 4° C. Wash the pellet with 70% ethanol (v/v). Let the pellet dry and resuspend in 350 μL of TE buffer. Add 350 μL phenol, shake vigorously and spin 5 min at 5000×g. The upper phase, containing the DNA, undergoes another phenol extraction. Followed four ether extractions, in order to get rid of phenol in the aqueous phase. Precipitate the DNA by adding 15 μL of 5M NaCl and 1 mL of ice-cold absolute ethanol. Wash the pellet with 70% (v/v) ethanol. Resuspend the dried pellet in a necessary TE volume to get a DNA concentration of 1 μg/μL.

Control of the Insertion Direction of the Fragment

The insertion direction of the fragment of interest in the vector is checked by sequencing, with the help of vector specific primers.
The surexpression of the SE is estimated by GST enzyme activity titration (see below).

Transformation of E. coli BL-21 with CaCl₂ Treatment

When ligation product are checked, E. coli BL21 cells can be transformed in order to get a significant sur-expression of SE. Make a 100 mL 2×TY culture of E. coli BL21, grow overnight at 37° C. Inoculate another 100 mL 2×TY with the E. coli preculture. Let grow at 37° C. until OD₆₀₀ₙₘ reach 0.5 to 0.7. Transfer the culture into a 50 mL tube. Spin at 3000 rpm 10 min at 4° C., discard the supernatant and add 10 mL 50 mM CaCl₂ to the pellet. Spin 10 min, 3000 rpm and at 4° C. and discard the supernatant. Repeat that step: add CaCl₂ and spin. Dissolve the pellet in 5 mL 50 mM CaCl₂. Transfer 150 μL of competent BL21 bacteria into a new eppendorf tube. Add 1 to 3 μL (2 ng) of transformed plasmid DNA. Transform bacteria by thermal shock method. Incubate 40 min at 4° C., then 2 min at 42° C. After 2 min chilling at 4° C., spread out the cells on a 2×TYA plate. Incubate 24 h at 37° C.
1.1. E. coli Production of Staphylococcal Enterotoxins (Apart from SED)

1.1.1. Preparation of Bacterial Lysate

Two 2 L flasks containing 400 mL of 2×TYA medium are inoculated with 50 mL of a BL21 E. coli preculture transformed by the pGEX-SE recombined strains, and incubated at 37° C. under 200 rpm shaking. When the OD₆₀₀ₙₘ reaches 0.4 to 0.6, fusion gene expression is induced with 0.2 mM IPTG at 25° C. overnight. Next day (18-22 h), bacteria are centrifuged at 5000×g during 10 min, at 4° C. The pellet is resuspended in 35 mL of PBS-EDTA 1 mM buffer. Bacteria cells are then lysed by French Press (French Pressure Cell Press, SLM AMINCO®) under a 600 bars pressure. Bacterial lysate is centrifuged during 30 min at 29000×g, at 4° C. in a Ti60 rotor (Beckman).

1.1.2. GST Enzyme Activity Titration

GST enzyme activity leads to an estimation of the fusion protein concentration in the environment. GST catalyzes a transfer reaction of the GSH on the CDNB (Chloro-1,2,4 Dinitrobenzène) [Habig and al., J. Biol. Chem., 249: 7130-7139, 1974] [20]. The product of the reaction absorbs at 340 nm whereas the free CDNB has its maximum at 270 nm. The kinetic occurs in a spectrophotometric bowl.

The enzyme buffer contains:

| | |
|---|---|
| H₂O (Milli Q) | 880 μL |
| × 10 reaction buffer (1M potassium phosphate, pH 6.5) | 100 μL |
| CDNB (100 mM in Ethanol) | 10 μL |
| Reduced Glutathion (100 mM in distilled water) | 10 μL |
| Sample or dilution 1/10, 1/20 | 10 μL |

The bowl is then placed into the spectrophotometer and the absorption evolution is measured at 340 nm every minutes during 5 minutes against a negative control, containing all the components apart from the 10 μL sample, replaced by 10 μL of distilled water (even without GST, there is a degradation of the substrate). This reaction is linear until A₃₄₀ₙₘ=0.8. The GST concentration must be adjusted in order to get a linear reaction during 5 minutes.

In those conditions, the GST concentration is proportional to the speed reaction (ΔA/min) in accordance to the following relation:

[GST]=vᵢ×k×1/d for 10 μL of sample

where:
[GST] in μg/mL
vᵢ=(ΔOD₃₄₀−ΔOD₃₄₀blanc)/min
1/d=dilution inverse
k=constant=545 μg·min·mL⁻¹
   k is determined from a commercial GST solution (Sigma) with a known concentration.

1.1.3. Purification of the Fusion Protein to GST, by GSH Affinity Chromatography

4 mL of gel Gluthathione Sepharose 4B GSH™ (GE Healthcare) (capacity of 20 mg of GST) is packed in a PD10 column (GE Healthcare). The column is equilibrated with 20 mL of buffer: PBS+EDTA 1 mM pH 7.5 per gravity. 16 mg equivalent GST (determined by the GST titration above) are applied in batch and left 30 min at +4° C. under smooth shaken. Then the gel is 10 min centrifuged at 200×g and at +4° C., and washed in PD10™ with 20 mL of PBS-EDTA.

6×2 mL fractions are eluted with the buffer: Tris 50 mM, GSH 30 mM, NaCl 500 mM pH8.0. Fractions with a significant DO₂₈₀ₙₘ (>0.2) are assembled, 10 μL (20 u) of PreScission Protease™ (GE Healthcare) are added to cleave the fusion protein and incubation one night at +4° C.

Note 1: in order to avoid contaminations, a different column for each enterotoxin should be used. Gels should also need to be washed with 10 mL of the solution: NaH₂PO₄ 30 mM, NaCl 2M pH6.5 between two runs.

Note 2: For SEG+8 and SEH+5, the sample may be applied a second time on the column GSH affinity, after the protease action and desalting against PBS, in order to get a maximum of GST; because those toxins are hardly separable of the GST, in the conditions of exchange ions chromatography presented below.

1.1.4. SEs Purification by Exchange Cations Chromatography

After the PreScission Protease™ action, the sample is diluted at 40 mL with 10 mM of start buffer containing 40 mM (MES or Hac) adjusted with NaOH (see table 5 below) and then dialyzed against that buffer during 2 h at +4° C., with a Spectra Porl™ membrane (Spectrum Laboratories) with a nominal molecular weight limit from 6 to 8000 Da. Then the sample is completed with 30 mM MES pH5.7 [from MES 500 mM pH5.7] and with 3 mM DTT [from DTT 1M]. After 5 min centrifugation at 5000×g at +4° C., different samples are applied on a 6 mL ReSource S™ or MonoS™ column (GE Healthcare), equilibrated in start buffer. After column washing, elution is made by a linear gradient of NaCl, supplied by the end buffer, i.e. start buffer+1M NaCl. The NaCl gradient slope goes from 0 to 300 mM maximum in 63 mL (flow rate 4 mL/min).

Note: between two runs and for each SE, the gel is washed with 2 mL de NaOH 0.5M.

TABLE 5

| | SEA + | SEB + | SEC1 + | SEE + | SEG + | SEH + | SEI + |
|---|---|---|---|---|---|---|---|
| | 5 | 5 | 8 | 5 | 8 | 5 | 5 |
| Start | MES | MES | MES | MES | Hac | Hac | MES |
| buffer | pH5.7 | pH6.1 | pH5.7 | pH5.7 | pH4.8 | pH4.8 | pH6.1 |
| Elution | 120 | 90 | 90 | 120 | 120 | 80 | 170 |
| (mM | | | | | | | |
| NaCl) | | | | | | | |

MES: 2-(N-Morpholino)ethane - sulfonic acid
Hac: acetic acid

### DTT: 1,4 Dithiothreitol

Note: Exchange ions separation shows different forms (enterotoxins found in several elution peaks), the prevalent form is kept.

Toxins are stored at −80° C. in aliquots of 2 mL maximum.

The purity is estimated in SDS-PAGE on a Phast System™ apparatus following the instructions of GE Healthcare. Briefly, 500 ng in 1 μL of SE (50 μL denatured in SDS 2.5% and β-mercaptoethanol) are applied on a PhastGel™ Gradient 10-15 and after electrophoretic migration, the gel is colored with blue coomassie (PhastGel Blue R).

### 1.2. SED Production

Some troubles appear upon the expression of the sed gene, that changed the protocole to an antigen purification from a referred strain of Staphylococcus aureus. SED is purified from S. aureus (87109 (FRI1157M) of the Institut Pasteur, Mrs Dr Debuyser collection):
BHI culture

From a preculture (6H), twelve 2 L flasks containing 200 mL of BHI are inoculated with 400 μL and incubated at 37° C. under 250 rpm overnight (15-18H). Bacteria are discarded by centrifugation at 7 000×g 10 min at +4° C.
Supernatant precipitation with ammonium sulfate

The supernatant is precipitated with solid ammonium sulfate (Sigma): 667 g to 1 L (90% of saturation) overnight (20-24H) at +4° C.
Capture by cations exchange chromatography at pH5.7 SP Sepharose FF gel

After centrifugation, the pellet is dissolved with 100 mL of water and dialysed in a SpectraPor1™ membrane (Spectrum Laboratories) first 2H at +4° C. against 4 L of water then against 2 L of MES 10 mM pH5.7. The sample is then completed to 30 mM MES pH5.7 with MES 500 mM pH5.7 solution and to 3 mM DTT with a 1M solution and centrifugated at 5000×g 5 min at +4° C. The sample is applied on a 50 mL SP Sepharose FF™ in a XK26 column (GE Healthcare) equilibrated with MES 40 mM+DTT 1 mM, pH5.7 buffer. After column washing, elution is made by direct application of a MES 40 mM+DTT 1 mM+300 mM NaCl, pH5.7 buffer.
Cations exchange chromatography at pH 5.7 Ressource S™ or MonoS™ column

After the first exchange chromatography, the sample is dialysed again in the same conditions as above. Then, it is applied on a 8 mL MonoS™ Tricorn column (GE Healthcare) equilibrated in the same start buffer as above. After washing, elution is made by a linear gradient of NaCl in the same end buffer. The NaCl gradient slope goes from 0 to 160 mM in 60 mL.
Hydrophobic interactions chromatography at pH 7.0 Source-Phenyl gel.

This step is delicate, SED highly hydrophobic is barely holded by the column. Now the pH of the sample is adjusted to 7.0 with a K₂HPO₄ 1.5M solution. Finally, the phosphate concentration of the sample is 350 mM (KH₂PO₄ 1.5M pH7.0) with 1.7M ammonium sulfate ((NH₄)₂SO₄ 3M pH7.0). The sample is applied on a 1 mL ReSource S™ or MonoS™ PHE column (GE Healthcare) equilibrated in KH₂PO₄ 50 mM+1.7M (NH₄)2SO₄+DTT 1 mM, pH7.0 buffer. After a short column washing with 3 mL, elution is made by a linear decreasing gradient of ammonium sulfate, supplied by the end buffer: KH₂PO₄ 50 mM+DTT 1 mM pH7.0. The gradient slope goes from 1700 mM to 0 in 10 mL.
Storage

Several runs of SED purification are put together (˜4 mg) and after dialysis, SED is concentrated on a MonoS column as above. The toxin is stored at −80° C. after checking the purity on SDS PAGE as above.

SED is recognized by the Oxoid SET RPLA™ kit (following the Oxoid instructions).

Enterotoxins A, B, C, D, E, G, H, and I are kept inside a box placed in a −80° C. freezer, located in a secured room. Amounts of enterotoxins stored and removed are recorded in a saved file and on a paper signed by the project responsible, all kept locked. A statement to the AFSSAPS for the B enterotoxin of Staphylococcus aureus is made. Every wild and recombinant microorganisms (even if they should be induced to get a toxin expression) are strictly controlled and used under a microbiological security post. Microorganisms used and toxins amounts which have to be used by the experience are denatured and sterilized, then confined on SharpSafe boxes before being evacuated to the Institute DASRI. Toxins amounts used and rejected are always less than 1 mg.

### Example 2

### Antibodies Anti-8SEs Origins

### 2.1. Procurement and Affinity Purification of the Antibodies

2.1.1. Generation of Toxoids

The 8 SEs (1 to 2 mg/mL) were turned into a toxoid by a 60 mM or 330 mM formalin treatment during 48H at 37° C. in buffer: 50 mM NaH₂PO₄+150 mM NaCl pH 7.5 [protocol inspired from the thesis of M. PIEMONT or inspirited from Gampfer and al. [Vaccine, 20: 3675-3684, 2002] [21]. The excess of formaldehyde was discarded by desalting on PD 10™ column (following the instructions of GE Healthcare) against PBS and the toxoids were stored at −20° C. During a second immunization drive, the 8 SEs were treated with 1% formol [inspirited from Gampfer, 2002, fore-mentioned] [21] in order to get bigger immune-serum stocks and antibodies for SEH and SEI with a better affinity. For each SE, two New-Zealand rabbits were injected in subcutaneous with 50 pg/500 μL (apart from SED: 25 μg then 100 μg) with the complete Freund's adjuvant to start and then the incomplete formulation was used.

2.1.2. Toxoids Preparations for Injection

Animals were immunized with 10 to 200 μg per rabbit (50 μg in a first generation of antibodies). Separately, the antigens were emulsified in a 1:1 mixture of complete or incomplete Freund's adjuvant (CFA or IFA) and PBS with a 2 mL syringe and a 1.1 mm needle. Rabbits were shaved off and the preparations were injected with 0.7 mm needles in three subcutaneous sites with 500 μL. Three booster immunizations were given at about 3-weeks intervals (for a second generation of antibodies IFA was used after the first injection).

2.1.3. Collect of Sera

Before the rabbits were bled, a sample of veinous blood from the ear was taken, about 500 μL. The sera was used in an Ouchterlony precipitation test. Briefly, in a plate with 0.6% agar in PBS, 40 μL of sera or 40 μL of SE at 150 μg/mL were put into 2 wells separeted from 1 cm. After overnight incubation at +4° C., a line of precipitation was considered like a hyper-immunisation. After that, rabbits were bled of 35 mL maximum under anesthesia: ketamin (20-40 mg/kg) and xylazin (3-5 mg/kg) [Borkowski and al., Clin. Tech. Small Anim. Pract., 14.(1): 44-49, 1999] [22] at the ear artery with a vacuum pump device. If necessary, the rabbits were killed with Dolethal™. After overnight blood clot retraction at +4° C., sera were centrifuged 5000×g 5 min and filtered (0.45 μm), and stored at −80° C. by 10 mL fractions.

Antibodies were purified by affinity on an activated “HiTrap NHS” support, where a native SE had been immobilized by injection-chromatography (Elution buffer: CitrateNa 0.1 M+NaCl 0.2 M, pH 2.5, then neutralization at pH 7.5 with Na₂CO₃ 1M).

2.1.4. Affinity Purification of Antibodies Anti-SE

The Aktä Purifier™ device (GE Healthcare) and Hi-TrapNHS gel 1 mL (GE Healthcare) were used wherein antigen corresponding to the antibody to purify was immobilized (see GE Healthcare protocol, “Ligand coupling procedure for HiTrap NHS-activated HP”) (see table 6 below).

TABLE 6

| | | |
|---|---|---|
| | Immobilized amount | |
| Ligand | (mg) | Date |

| | | |
|---|---|---|
| SEA | 6.7 | 08/02/2008 |
| SEB | 5.4 | 26/10/2007 |
| SEC1 | 3 | 02/04/2008 |
| SED | 4 | 15/04/2010 |
| SEE | 6.8 | 14/03/2008 |
| SEG | 5.8 | 23/04/2008 |
| SEH | 8.5 | 28/03/2008 |
| SEI | 5.3 | 28/01/2008 |

The column was equilibrated with 10 mL of PBS with a flow rate of 1 mL/min. The serum provided by the hyper-immunized rabbit, was centrifuged 5 min at 5000×g, and 5 mL of serum were applied at the flow rate of 0.5 mL/min. The column was washed with PBS. The antibodies were evaluated with citrate buffer [0.2M NaCl, 0.1M citric acid, pH 2.5], at a flow rate of 1 mL/min. Fractions were then neutralized to pH 8 with 1M Na₂CO₃ pH 9.5. The OD₂₈₀ₙₘ was measured. Fractions with a significant OD₂₈₀ₙₘ (>0.2) were assembled. Antibodies were then dialyzed against 2 L of 30 mM NaH₂PO₄, 100 mM NaCl, pH 7.3 at 4° C. overnight.

The antibodies purified by affinity were frozen and stored at −80° C. at a minimal concentration of 1 mg/mL.

### 2.2. Specificity of Affinity Purified Antibodies and Achievements of Mono SE-Specific Antibodies

2.2.1. DOT-BLOT

A piece of nitrocellulose (Protan^{BA83™ 0.22} μm Schleicher et Schuell) of about 7×3 cm was cut. 1 μL, i.e. 10 ng, of each SE (stock solutions at 10 μg/mL) and a blank (BSA) were put and left to dry several minutes. The membrane was drenched (˜20 mL) in the blocking buffer [PBS, 5% skimmed milk(w/v)], left at 4° C. overnight, then washed with PBS+0.05% Tween20 5 min by immersion. The immunoabsorbed Ab antiSE, which the specificity is to check, was added at 1 μg/mL in 20 mL of PBS+0.05% Tw20+1% skimmed milk and left 2 h under smooth shaken, then washed 3 times. Goat Ab-POD anti-rabbit IgG purified by affinity (Sigma A6154), was added at a 1/800 dilution in the same buffer that primary Ab and incubated in the same conditions, then washed 3 times. The reaction was revealed according to the Opti 4 CN™ kit instructions (BioRad): 9 mL of water, 1 mL of the kit buffer and 0.2 mL of the kit 4 CN, for 30 min max., depending on the blank. The membrane was washed with water several times and finally scanned.

2.2.2. Depletion of Antibodies Already Affinity Purified

The HiTrap™ gel (1 mL) with an antigen (namely an immunization-unrelated staphylococcal enterotoxin) immobilized responsible for the cross reaction was chosen. The gel was equilibrated with 5 to 10 mL of PBS on AktaPurifier™ device (GE Healthcare) at flow rate 1 mL/min. An amount of Ab was applied from one run of affinity chromatography (1 mL of gel), at a flow rate 0.5 mL/min, and Ab which passed through the immunoabsorption column were collected. Ab eluated by the citrate buffer were removed, about 10 mL at flow rate 1 mL/min. The column was washed with 5 to 10 mL of PBS. The column was stored in PBS+NaN3 0.1% at 4° C. After control of their specificity (by DOT-BLOT) against the immunogen and/or cross reactivity with an antigen (namely an immunization-unrelated staphylococcal enterotoxin), depleted Ab were stored at −80° C. at a concentration of 1 to 3 mg/mL.

If a cross reaction was still detected, the depletion step was again implemented as above described against the same antigen or another antigen as above defined (which is still not the immunogen) until obtaining the monospecificity of depleted Ab. The order of successive columns could be determined by one skilled in the art according to the extent of each cross reaction detected, namely from the stronger cross reactivity to the weaker cross-reactivity.

Step-by-step immunoabsorptions/depletions in the exact order of the successive columns made on the affinity purified antibodies are shown in the table 7 below.

Note 1: For IgG, DO₂₈₀=1.0 means a concentration of 750 μg/mL.

Note 2: Concentration of Ab is possible by ultrafiltration, using Amicon® 4 mL or 15 mL, cut off 10 kDa units (according to Millipore™ instructions).

TABLE 7

| Ab > SEA | Ab > SEB | Ab > SEC1 | Ab > SED | Ab > SEE | Ab > SEG | Ab > SEH | Ab > SEI |
|---|---|---|---|---|---|---|---|
| E E I B D | C1 C1 G | B B G | A A E | A A I C1 | A B I C1 | B D | E C1 G B A |

Those depleted antibodies were frozen in the buffer: NaH₂PO₄ 30 mM+NaCl 100 mM, pH 7.3.

Such antibodies may be used downstream for several kinds of applications (beads-based multiplex technologies, chip-based protein microarray, immunocapture coupled to MALDI-TOF, etc. . . . ).

### Example 3

### Coupling Of Anti-SEs Antibodies to Luminex Microspheres and Biotin

3.1. Beads Sensitization with the Depleted Antibodies Anti-SEs

Each depleted antibody anti-SEs was combined with a microsphere of an unique color-code according to the table 8 below.

3.1.1 Protocol (Inspired from Amine Coupling Kit of BioRad Ref 171-406001)

For each SE, 19.5×10⁶ carboxylated beads were activated at pH6.0 (Buffer: MES 0.1 M+NaCl 0.15 M pH 6.0) by 50 mg/mL of EDC with sNHS during 30 minutes under shaking at room temperature. After wash by centrifugation 10 min at 10 000×g, 360 μg of depleted antibodies (without NaN₃ and Amine) were added, i.e. 18.5 μg for 1 million of beads, and were incubated in PBS during 2H under shaking, at room temperature and in darkness. After wash, free sites were blocked with ethanolamine (Buffer: ethanolamine 50 mM+NaCl 150 mM, pH 8.5) during 30 minutes. Then the blocking buffer was replaced by the storing one: PBS+NaN₃ 0.05% (p/v). The storage was made at +4° C. and in darkness during several months after assembling in one fraction the 8 types of beads at 1×10 beads of each color region/mL.

Finally, the fluorescence of the coupled beads was measured by the BioPlex-100™ with biotin-coupled goat antigen (Sigma), rabbit anti-IgG and SEPE. The final suspension concentration was determined by a 1 mm³ hemacytometer.

3.1.2. Acquired Results for the Validation of the Beads Set

TABLE 8

| | Beads | | | Coupling | |
|---|---|---|---|---|---|
| | COOH | | [beads]/ | yield in % | SEPE |
| | color- | Coupling | mL | for the | control |
| Ab anti | region | date | in 1.8 mL | beads | in UFI |
| SEA | 8 | 09/02/2010 | 7.77 · 10⁶ | 72 | 22 798 |
| (E E I B D) | | | | | |
| SEB | 12 | 10/02/2010 | 9.06 · 10⁶ | 84 | 24066 |
| (C1 C1 G) | | | | | |
| SEC1 | 16 | 10/02/2010 | 8.95 · 10⁶ | 83 | 21665 |
| (B B G) | | | | | |
| SED | 20 | 23/04/2010 | 7.53 · 10⁶ | 70 | 23873 |
| (A A E) | | | | | |
| SEE | 30 | 19/02/2010 | 6.81 · 10⁶ | 63 | 14436 |
| (A A I C1) | | | | | |
| SEG | 34 | 12/02/2010 | 6.48 · 10⁶ | 60 | 8489 |
| (A B I C1) | | | | | |
| SEH | 38 | 12/02/2010 | 8.78 · 10⁶ | 81 | 7050 |
| (B D) | | | | | |
| SEI | 42 | 19/02/2010 | 7.74 · 10⁶ | 71 | 7949 |
| (E C1 G B A) | | | | | |

According to the supplier BioRad, a bead was sufficiently coupled if UFI SEPE>2000 and UFI blank<100. This was the case for the 8 types of beads.

### 3.2. Biotin Coupling of Antibodies

Like the secondary antibodies, the simply affinity purified antibodies (8AbBt(X)) individually coupled to biotin were used. The test specificity was made by the primary antibody immobilized on the beads, since it ensured the antigens selection and all the test specificity.

The 8 depleted Ab individually biotin-coupled (AbBt(−)) were also used for the need of the test specificity (see below). That specificity was compared to the one of AbBt(x); the specificity deviation was not significant, but the sensibility seemed to be lightly strengthened by the use of affinity purified antibodies, for a low production cost.

Protocol of Biotin Coupling:

The protocol was unchanged whatever the Ab chosen (without NaN₃ and Amine). 200 μg of Ab at 1 mg/mL in PBS were mixed to 10 μL of sNHS-Lc-Bt (Molecular Probes) at 2.67 mM during 2H at room-temperature, i.e. a initial molar ratio [Bt]/[Ac]=20. Free biotin was blocked by 10 μL of ethanolamine 100 mM, pH 7.5 during 30 min at room-temperature, then 5.5 μL of NaN₃ were added at 2%, i.e. a final concentration of 0.05% (p/v). The AbBt(−) and the AbBt(x) on either side were assembled in 2 fractions (x) and (−) only after the specificity test (see below). Each fraction was aliquoted and stored at −20° C. at 100 μg/mL.

### Example 4

### General Instrumental Protocol: Luminex xMAP Technology

As a preamble, a reminder of the protocol of BioPIex100™ utilization:
A plan of the 96-wells plate for a maximum of 31 samples was drawn, made in duplicate (with 1 blank for each type of cheese matrix), 12 standards (from 16384 to 8 pg/mL, 2 in 2 dilution), 3 identical standard blanks and 2 controls at 4000 and 400 pg/mL of the 8 SEs (those two points could be replaced by external control of the laboratory). Every points were in duplicate.
Antigen aliquot was defrosted.
From the stock suspension of beads coupled to the 8 depleted Ab anti-SEs at 1. 10⁶ beads/region/mL, a final solution at 10⁶ was prepared and 50 μL were splitted out in a 96-wells multiscreen plate (Millipore), i.e. 5000 beads/region/well.
Washing by aspiration with the manifold pump: 2 to 3 inches Hg of depression then 100 μL of TBS (Tris-HCl 40 mM+NaCl 140 mM, pH 7.5) were added and re-aspirated.
50 μL/well of antigen (sample, standard, blank and control) were transferred according to the plate plan and incubated under high shaking (600) during 1 h30, at room-temperature and in darkness.
Then washed 3 times by aspiration.
50 μL/well of the 8Ab-antiSEs-Bt(x) solution (non depleted) were added at 0.25 μg/mL in TBS and incubated 1 h, at room-temperature and in darkness.
Then washed 3 times by aspiration.
50 μL/well of 0.5 μg/mL SEPE solution (stock solution at 1 mg/mL, Molecular Probes réf: S866) were added and incubated 15 min, at room-temperature and in darkness.
Then washed 3 times by inspiration.
Each well was resuspended in 125 μL of TBS+Tween 20 at 0.05% and stirred 1 min.
The plate was read at the BioPIex100™ according to the BioRad instructions (calibration and start up).
Results were analyzed for each SE: the assays concentration was determined after subtraction of the cheese matrix blank, with the aid of the inverse function f−1 which is:

$$X = ( {( \frac{( a - d )}{( y - d )} )}^{{g}^{- 1}} - {1}^{{b}^{- 1}} ) \times c$$

x=concentration
y=response (FI)
a=estimated response at infinite concentration
b=slope of tangent at midpoint
c=midrange concentration or midpoint
d=estimated response at zero concentration
g=asymmetry factor

Note: Beads concentration (5000/SE/well) is chosen for a plate reading of 30 to 45 min. The sensibilization with 18.5 μg of Ab/million of beads produces a satisfactory maximal signal (>2000 UFI), apart from SEH and SEI which have a lower signal. 8 AbBt(x) and SEPE concentration produce the lowest limit of quantification (LOQ).

### Example 5

### Titration Test Validation

Results exposed there concern the xMAP technology application for the 8SEs titration besides of cheese matrix.

### 5.1. Specificity

The aim was to estimate the degree of cross-reaction between the primary depleted Ab coupled to beads on one hand, and the secondary biotin-coupled Ab in the other hand, towards the 8 SEs.

5.1.1 Modification of the Protocol Exposed in EXAMPLE 4.

In that aim, those following ELISA sandwiches were made up:
A: beads 8Ab-antiSEs (depleted)+1 SE/well+8AbBt(x)+SEPE, with the Ag at about 19.6 ng/mL, a concentration equal to the 51 point (16.4 ng/mL) of the standard range (table 9).
B: beads 8Ab-antiSEs (depleted)+8 SE/well+1AbBt(x)+SEPE (table 10).

For results analyzing, the net value of fluorescence (Fl-blk) was noticed every time. The specificity, expressed in %, was given by the ratio of the detected signal (Fl-blk) by the specific signal for each SE.
C: beads 8Ab-antiSEs (depleted)+1 SE/well+8AbBt (depleted)+SEPE, with the Ag at about 19.6 ng/mL, a concentration equal to the S1 point (16.4 ng/mL) of the standard range (table 11).

5.1.2. Specificity—Results—Conclusions

TABLE 9
ELISA specificity: A

| % | SEA | SEB | SEC | SED | SEE | SEG | SEH | SEI |
|---|---|---|---|---|---|---|---|---|
| antiSEA | 100 | 0 | 0 | 0 | 0 | 0 | 0 | 0 |
| (E E I B D) | | | | | | | | |
| antiSEB | 1 | 100 | 0 | 0 | 0 | 0 | 0 | 0 |
| (C1 C1 G) | | | | | | | | |
| antiSEC1 (B B G) | 1 | 0 | 100 | 0 | 0 | 0 | 0 | 0 |
| antiSED (A A E) | 0 | 0 | 0 | 100 | 0 | 0 | 0 | 0 |
| antiSEE | 0 | 0 | 0 | 0 | 100 | 0 | 0 | 0 |
| (A A I C1) | | | | | | | | |
| antiSEG | 1 | 0 | 0 | 0 | 0 | 100 | 0 | 0 |
| (A B I C1) | | | | | | | | |
| antiSEH (B D) | 2 | 0 | 1 | 1 | 0 | 0 | 100 | 0 |
| antiSEI | 0 | 0 | 0 | 0 | 0 | 0 | 0 | 100 |
| (E C1 G B A) | | | | | | | | |

Conclusion: the 8 depleted Ab anti-SEs did not produce significant cross-reaction between the 8 SEs. A depleted Ab immobilized to a bead, only fixed its own antigen.

TABLE 10
ELISA specificity: B

| % | SEA | SEB | SEC | SED | SEE | SEG | SEH | SEI |
|---|---|---|---|---|---|---|---|---|
| antiSEA Bt(X) | 100 | 2 | 5 | 1 | 62 | 3 | 2 | 7 |
| AntiSEB | 2 | 100 | 70 | 0 | 1 | 1 | 0 | 3 |
| antiSEC1 | 3 | 66 | 100 | 0 | 4 | 12 | 0 | 3 |
| antiSED | 2 | 0 | 0 | 100 | 1 | 0 | 1 | 0 |
| antiSEE | 64 | 0 | 1 | 3 | 100 | 1 | 1 | 42 |
| antiSEG | 32 | 26 | 47 | 0 | 34 | 100 | 5 | 29 |
| antiSEH | 0 | 2 | 0 | 0 | 0 | 0 | 100 | −1 |
| antiSEI | 1 | 0 | 0 | −1 | 24 | 0 | −5 | 100 |

Conclusion: B ELISA revealed cross-reactions with some SEs for the 8 simply affinity purified Ab anti-SEs. However, as the bead-immobilized depleted Ab were strictly specific, the simply affinity purified Ab as secondary Ab could be used. B ELISA also suggested a different immunoabsorption protocol from the one made up with the DOT BLOT results.

TABLE 11
ELISA specificity: C

| % | SEA | SEB | SEC | SED | SEE | SEG | SEH | SEI |
|---|---|---|---|---|---|---|---|---|
| antiSEA | 100 | 0 | 0 | 0 | 1 | 0 | 0 | 0 |
| (E E I B D) | | | | | | | | |
| antiSEB | 0 | 100 | 0 | 0 | 0 | 0 | 0 | 0 |
| (C1 C1 G) | | | | | | | | |
| antiSEC1 (B B G) | 1 | 0 | 100 | 0 | 0 | 0 | 0 | 0 |
| antiSED (A A E) | 0 | 0 | 0 | 100 | 0 | 0 | 0 | 0 |
| antiSEE | 0 | 0 | 0 | 0 | 100 | 0 | 0 | 0 |
| (A A I C1) | | | | | | | | |
| antiSEG | 1 | 0 | 0 | 0 | 0 | 100 | 0 | 0 |
| (A B I C1) | | | | | | | | |
| antiSEH (B D) | 1 | 0 | 0 | 0 | 0 | 0 | 100 | 0 |
| antiSEI | 0 | 0 | 0 | 0 | 0 | 0 | 0 | 100 |
| (E C1 G B A) | | | | | | | | |

Conclusion: identical to the table A conclusion.

Note: That type of specificity test did not announce any specific cross-reaction in cheese matrix, like with protein A. Such interactions with protein A could easily be circumvent by the addition of 10 μg/mL aspecific rabbit IgG.

### 5.2. Sensibility and Detection Limits

Aim: from the standard curve of the antigens titration, deduce the LOD (limit of detection) and the LOQ (limit of quantification).

5.2.1. Standard Curve

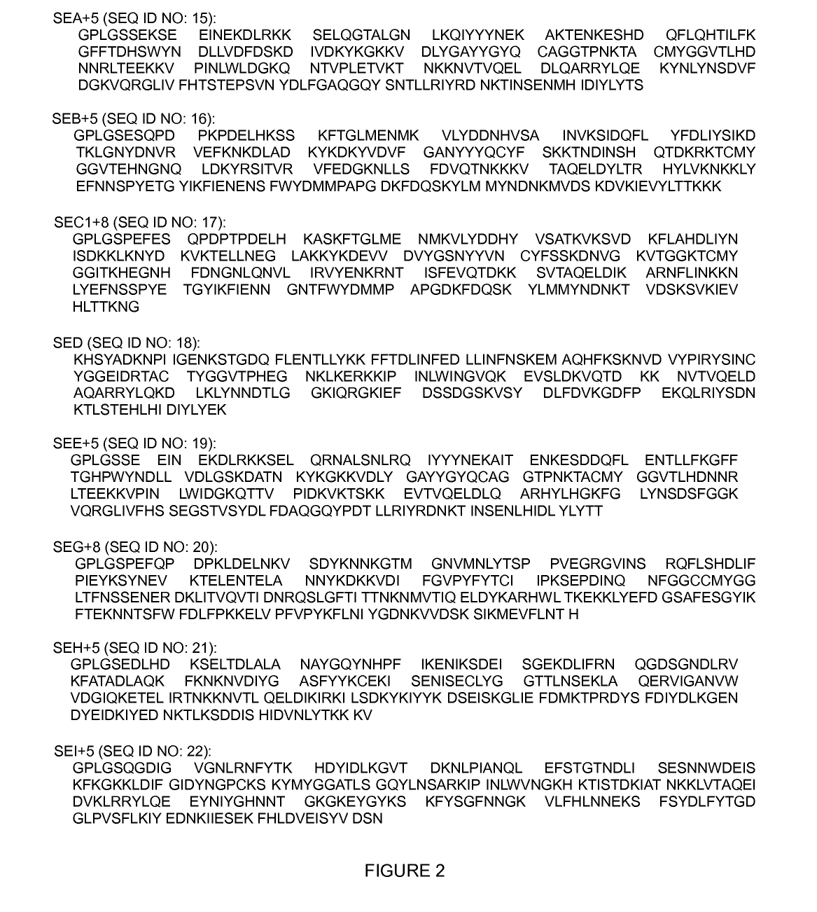

The reference concentrations (Cref) of the 8 SEs were obtained from spectrophotometry at DO₂₈₀ₙₘ according to the Beer-Lambert law. The extinction coefficients (ε) used were those given by the server software http://expasy.org as Protparam (see table 12 below), according to the corresponding primary sequences (FIG. 2):

TABLE 12

| SE | SEA | SEB | SEC1 | SED | SEE | SEG | SEH | SEI |
|---|---|---|---|---|---|---|---|---|
| ε en L.g⁻¹ · cm⁻¹ | 0.725 | 0.779 | 0.915 | 1.021 | 0.736 | 0.912 | 0.964 | 0.727 |

1 mL of a stock solution of the 8SEs at 6.55 μg/mL has been prepared in TBS and frozen at −40° C.

In TBS+Tween20+IgAasp [aspecific IgG, i.e. prepared from a non-immunized rabbit serum and affinity purified on Sepharose Fast Flow Protein G™ (GE healthcare)] Tris 40 mM+NaCl 140 mM pH 7.5+Tween 20 at 0.05%+aspecific rabbit IgG at 10 μg/mL, purified on G protein (stock solution at 5200 μg/mL).

The stock solution of SEs was diluted at 1/100 with the same buffer, then a cascade dilution, 2 in 2 between 65.6 and 0.008 ng/mL. The standard range used was from 16.4 to 0.008 ng/mL, i.e. 12 points of measure.

A non linear Logistic-5PL regression was made with StatLIA™ Immunoassays software of Brendan Scientific, included in BioPlex Manager 4.0.

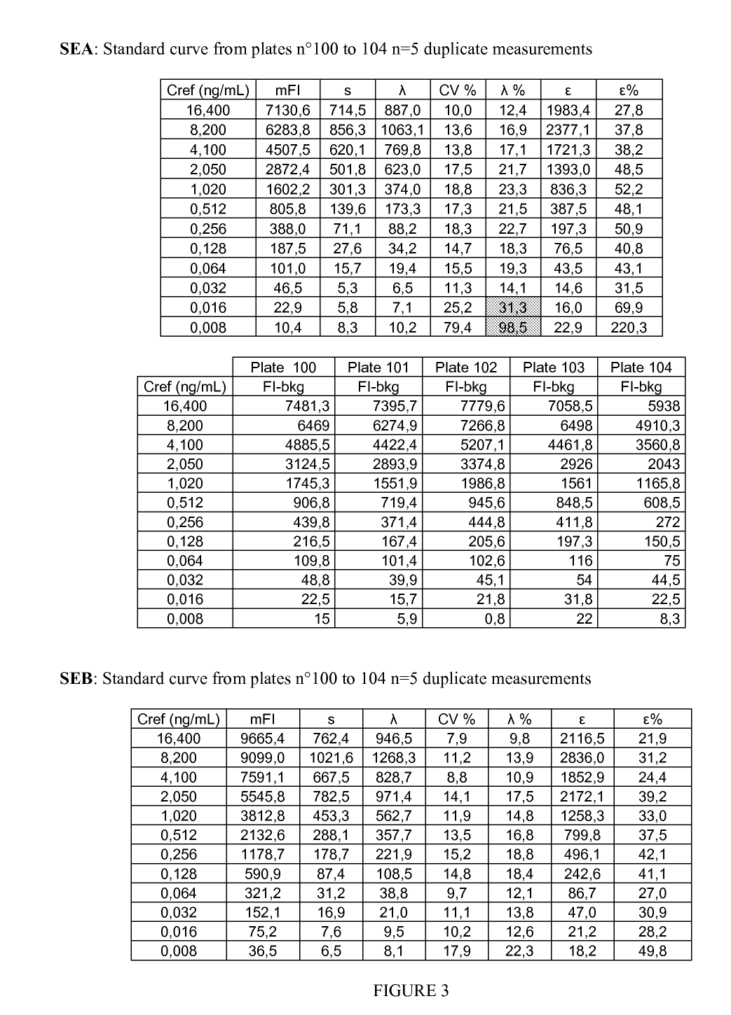

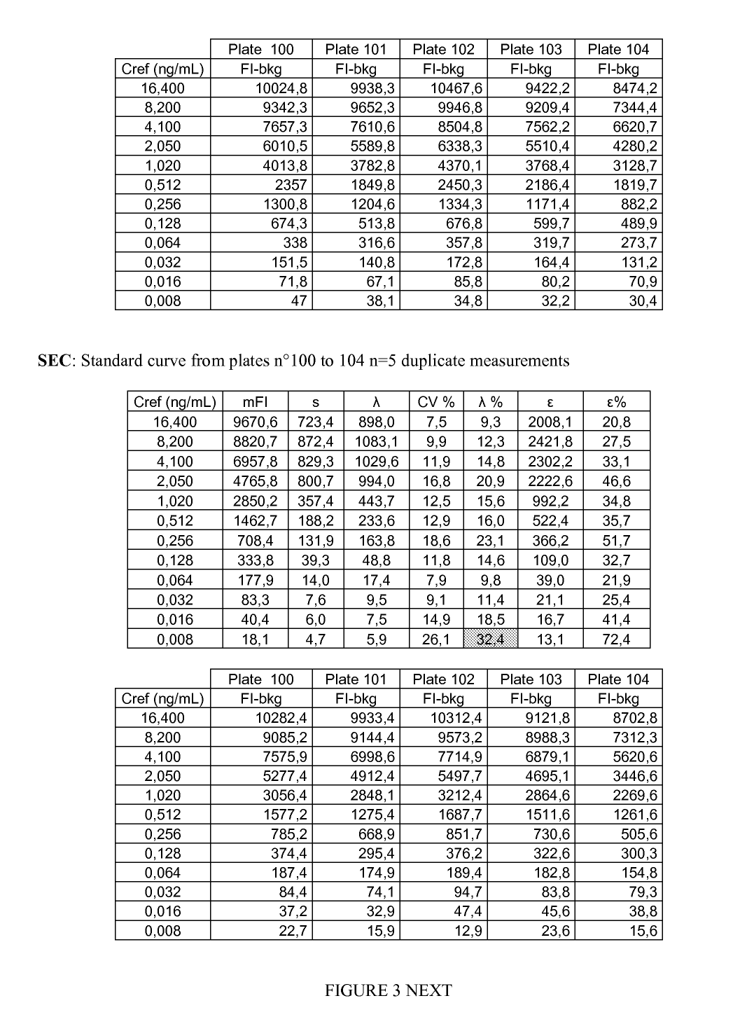

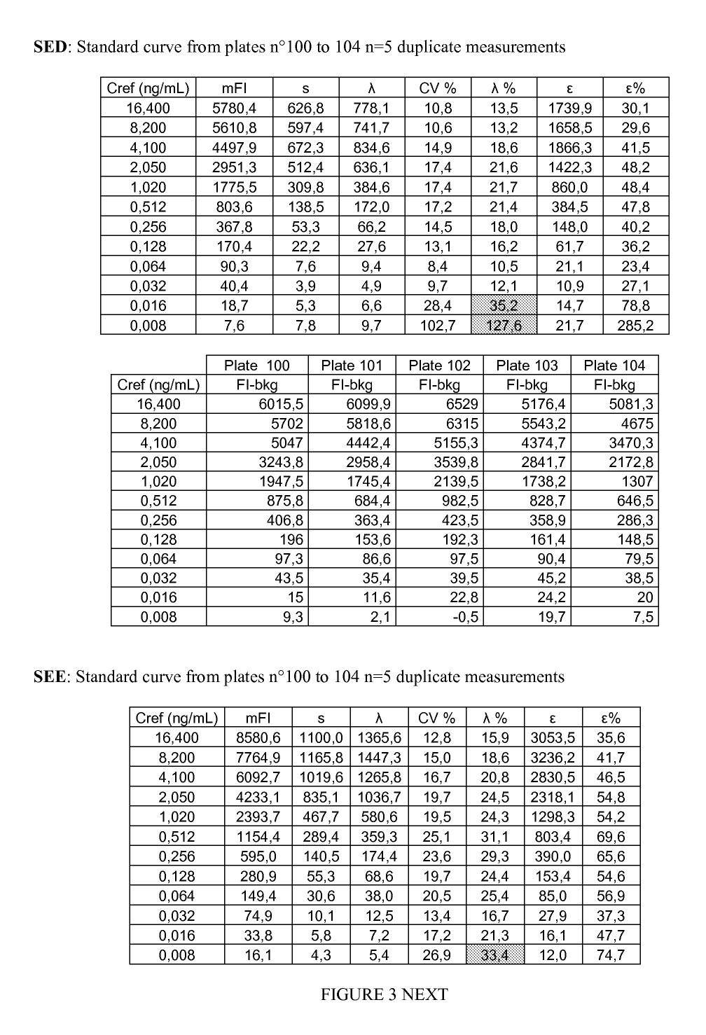

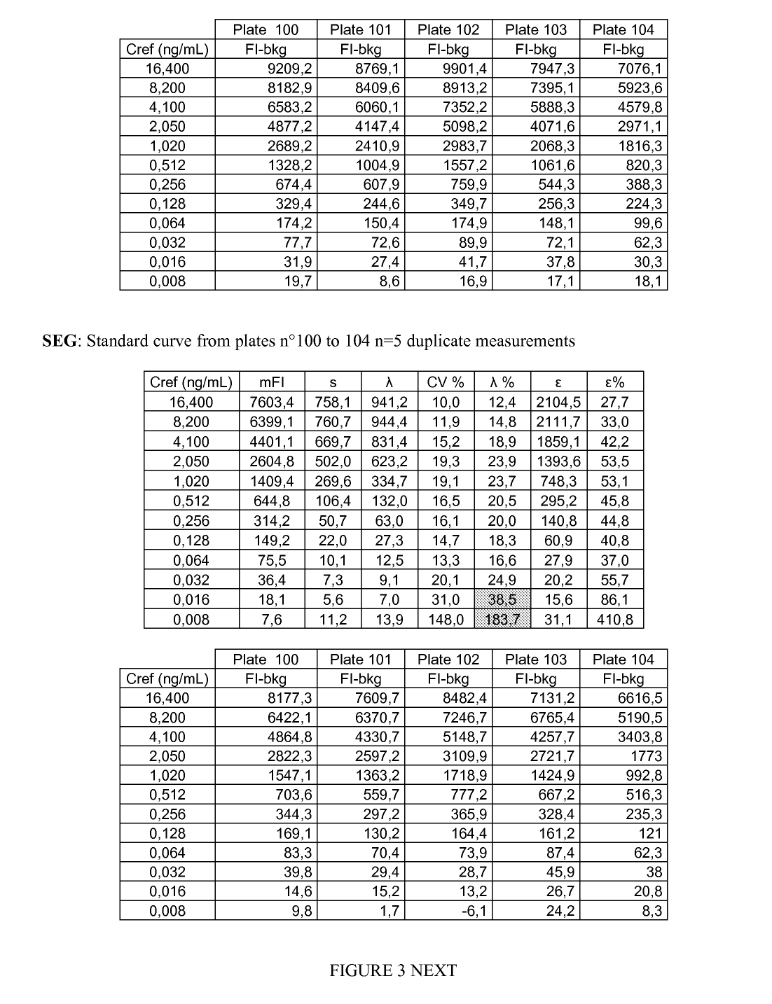

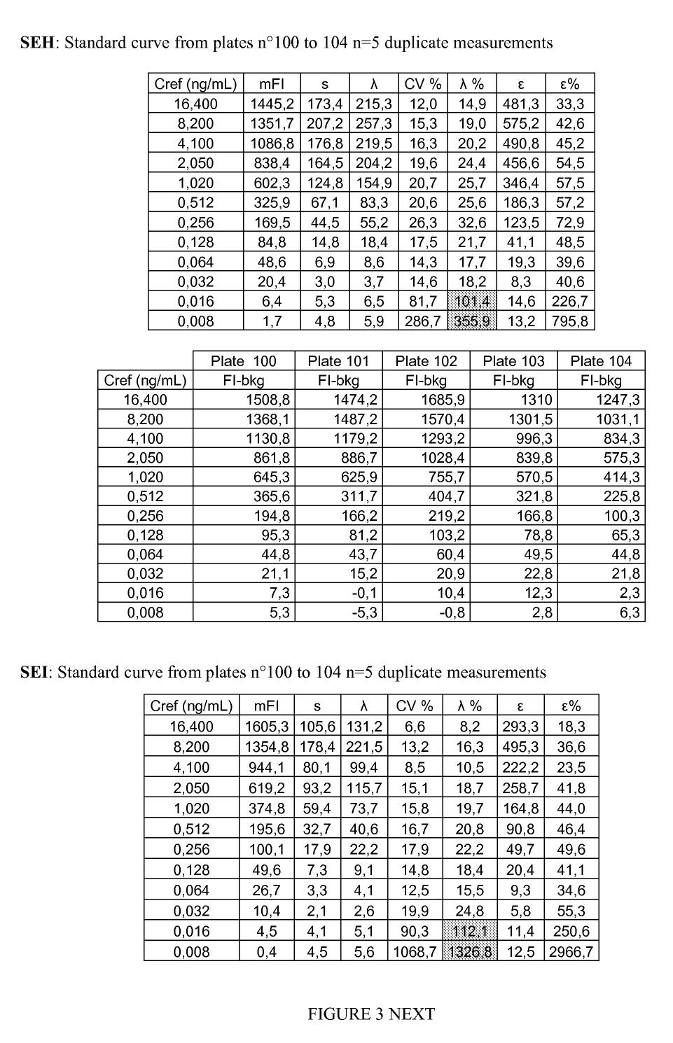

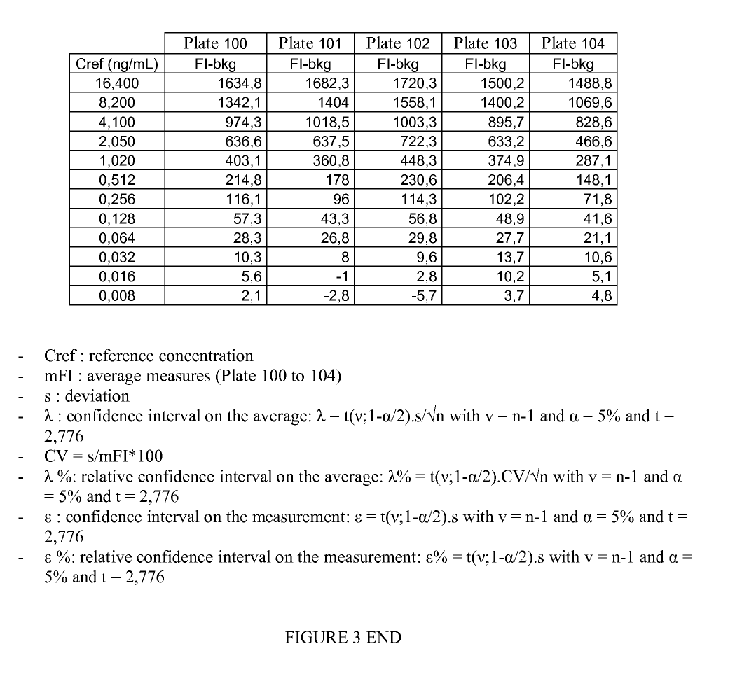

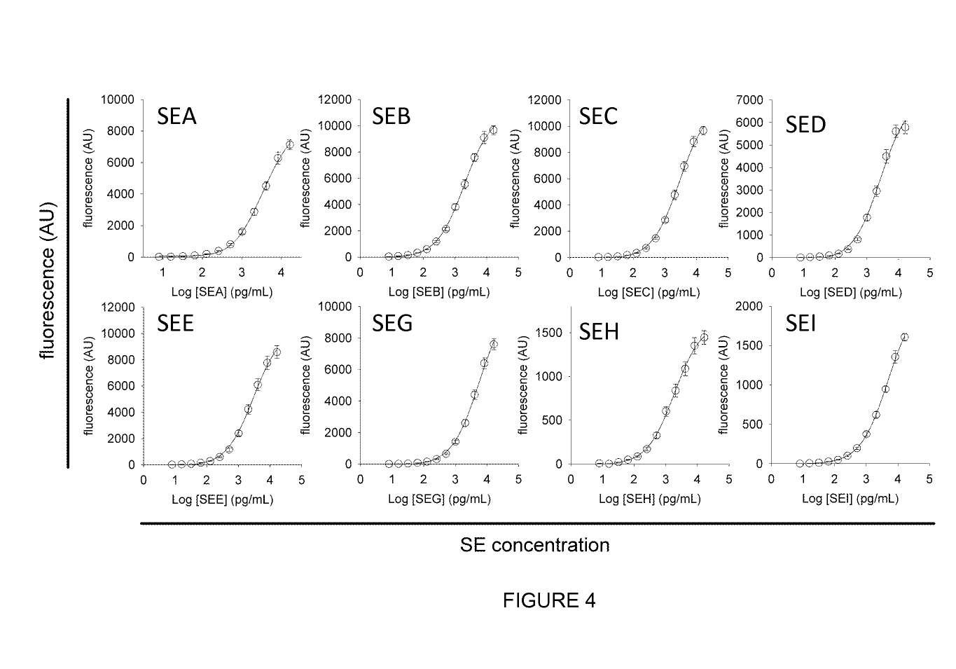

Results: Standards curves: Observed concentrations (Cobs) (Plates 100 to 104, i.e. n=5) see FIGS. 3 and 4: “averaged standard curves and series of assays 100 to 104.

In theory, after several types of regression tested (linear, exponential, logistic, . . . ), the type kept was the one that reflected the best the measured magnitude (y) depending of the explanatory variable (x). If the 12 points of the standard range of the test plates (100 to 104) were linked by two types of regression: one linear and the other non linear Biologistic-5PL; the look of the non linear Biologistic 5-PL regression explained better the y=f(x) evolution than a straight line.

5.2.2. Calculation of LOD (Limit of Detection), Lower LOQ (Limit of Quantification) and AWR (Assay Working Range).

The aberrant points among the 6 blank wells were removed with the Grubbs test.

LOD and lower LOQ were determined according to Soejima and al. [Int. J. Food Microbiol., 93(2): 185-194, 2004] [23] (see table 13).

If d was the estimated response at zero concentration and S the blanks standard deviation (n=6), then LOD=f−[(d+3.3*S), with f−1 the inverse function of Fl=f(conc), provided by StatLIA, and lower LOQ=f−1(d+10*S).

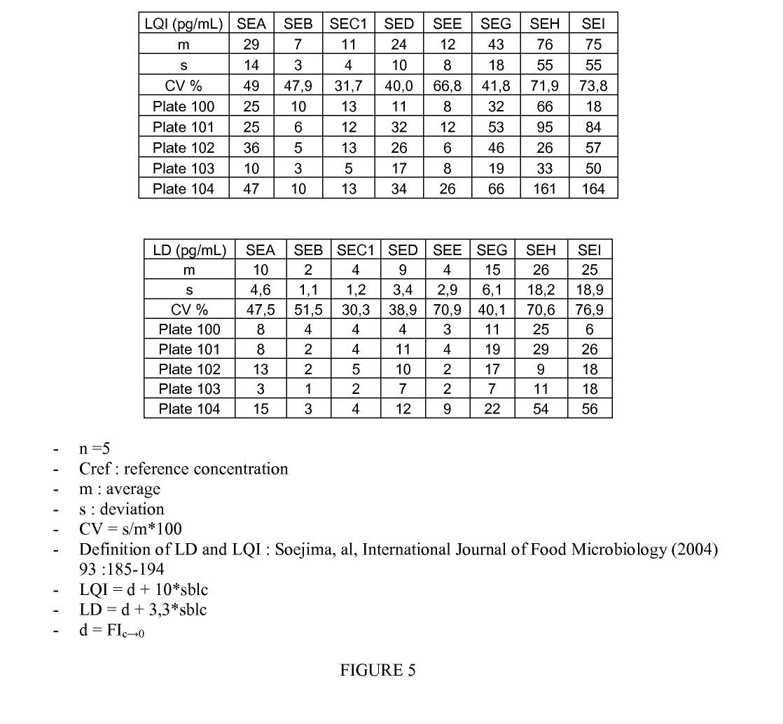

Results (plate 100 to 104, i.e. n=5) (see FIG. 5):

TABLE 13

| pg/mL | SEA | SEB | SEC1 | SED | SEE | SEG | SEH | SEI |
|---|---|---|---|---|---|---|---|---|
| LOD | 10 +/− | 2 +/− | 4 +/− | 9 +/− | 4 +/− | 15 +/− | 26 +/− | 25 +/− |
| | 4.6 | 1.1 | 1.2 | 3.4 | 2.9 | 6.1 | 18.2 | 18.2 |
| Lower | 29 +/− | 7 +/− | 11 +/− | 24 +/− | 12 +/− | 43 +/− | 76 +/− | 75 + /− |
| LOQ | 13.9 | 3.2 | 3.6 | 9.7 | 8.1 | 18.1 | 54.8 | 55.1 |

That method did not include a higher limit of quantification, and was dependent of the blanks (base line).

5.3. Linearity of the Titration: Cobs ═F(Cref)

Linearity of Cobs=f(Cref) tested on the 12 points of the standard range (n=5). Since R²>0.99, the response could be considered as linear for indicated values (see the table 14 below).

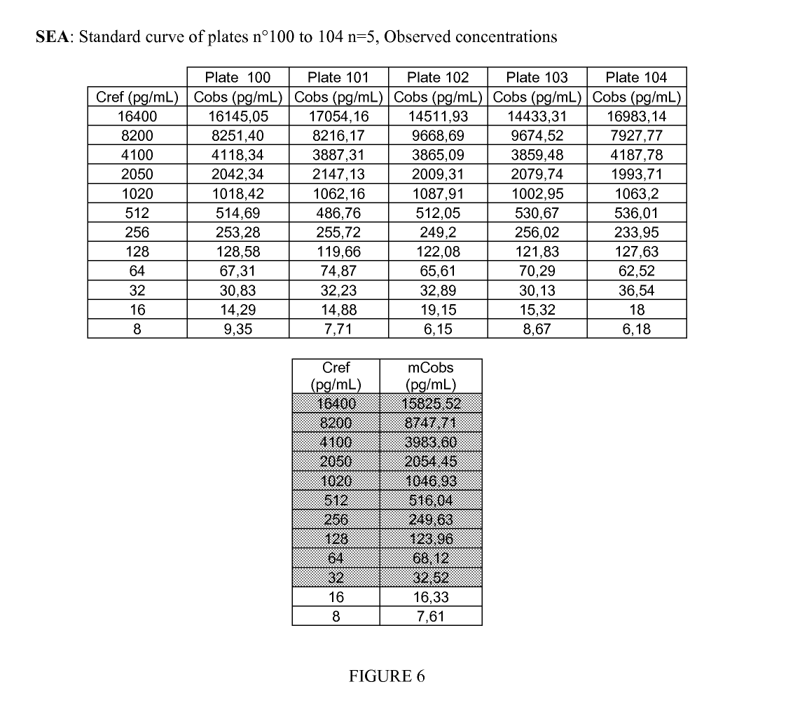

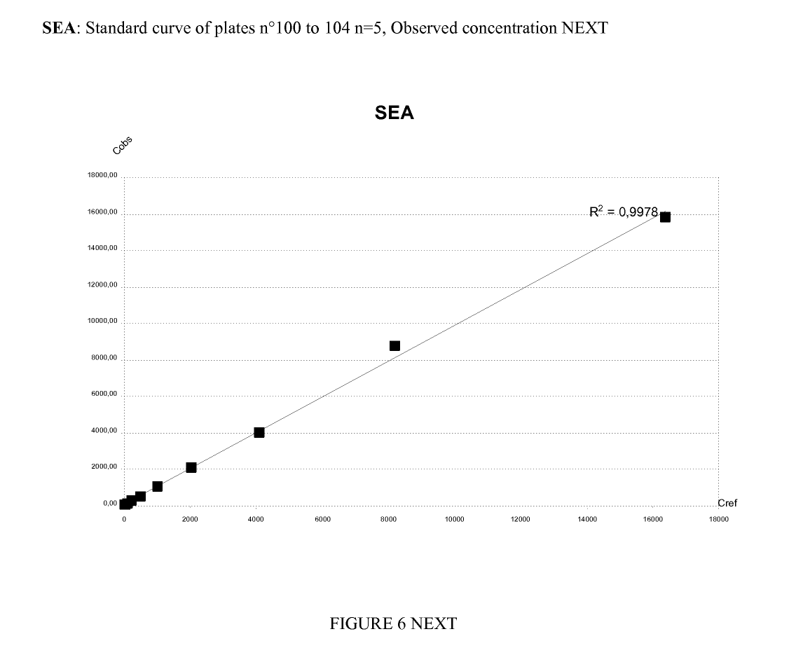

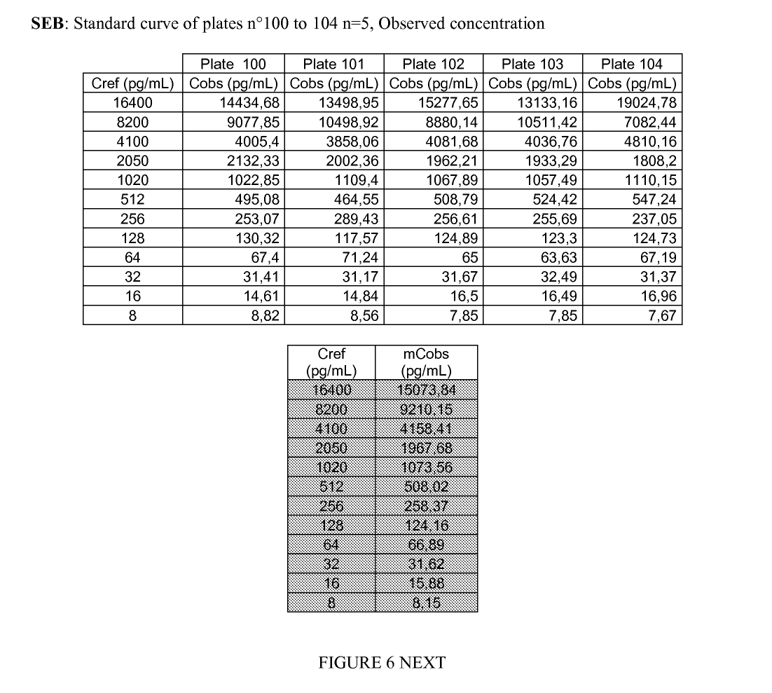

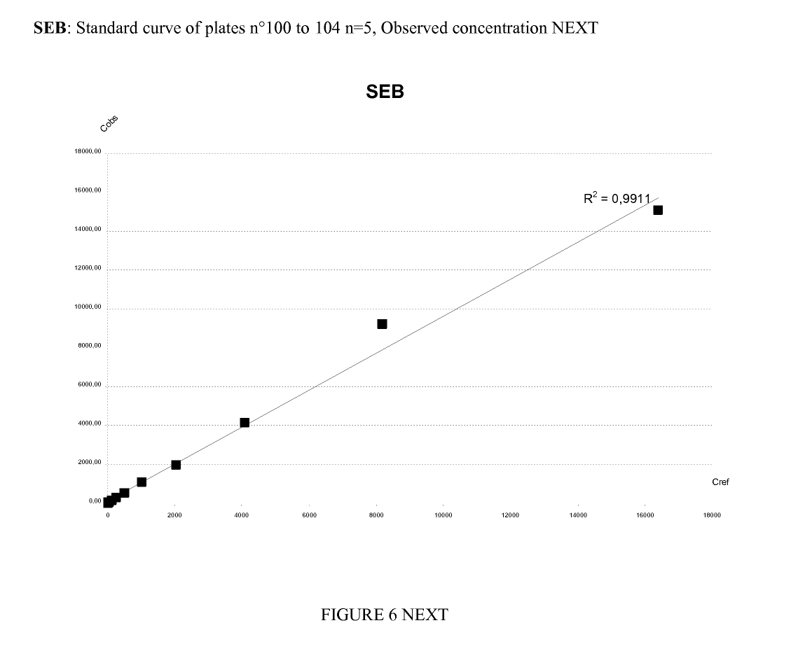

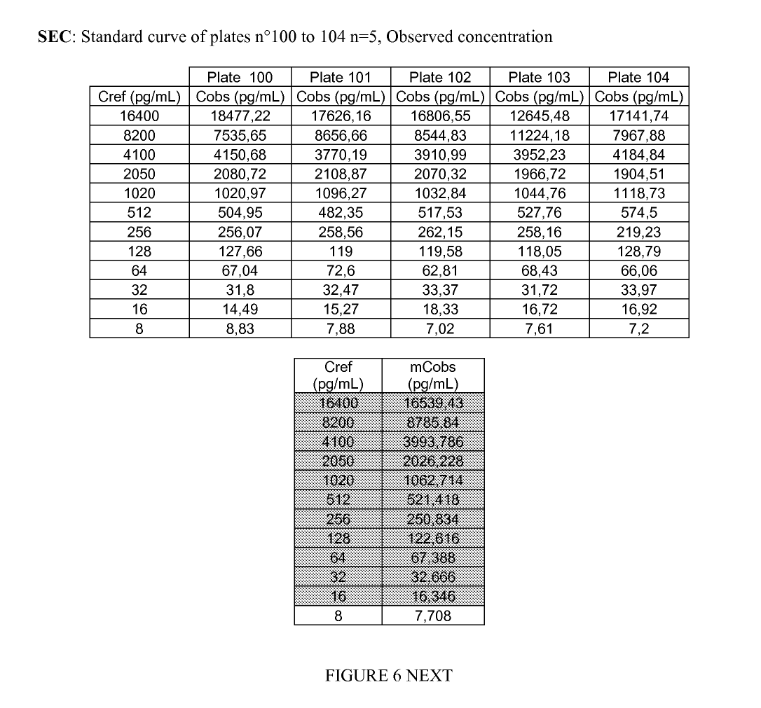

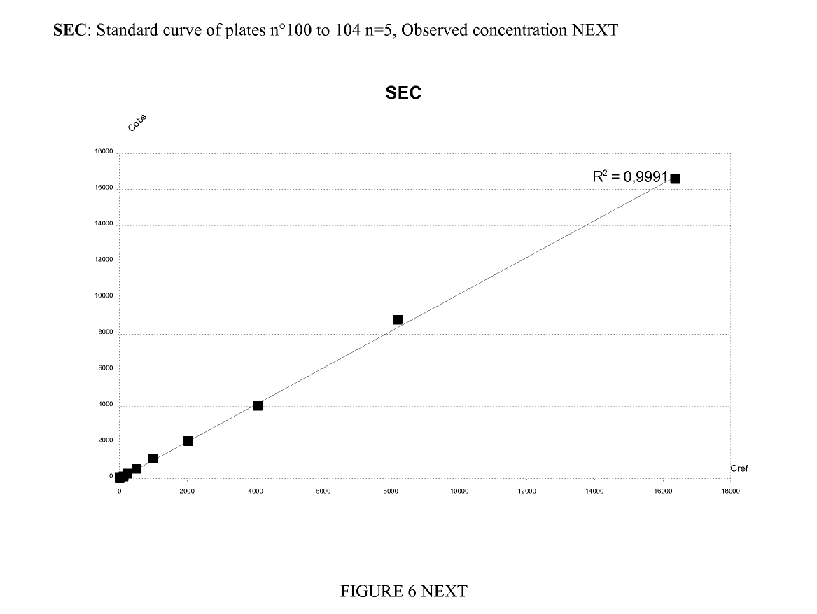

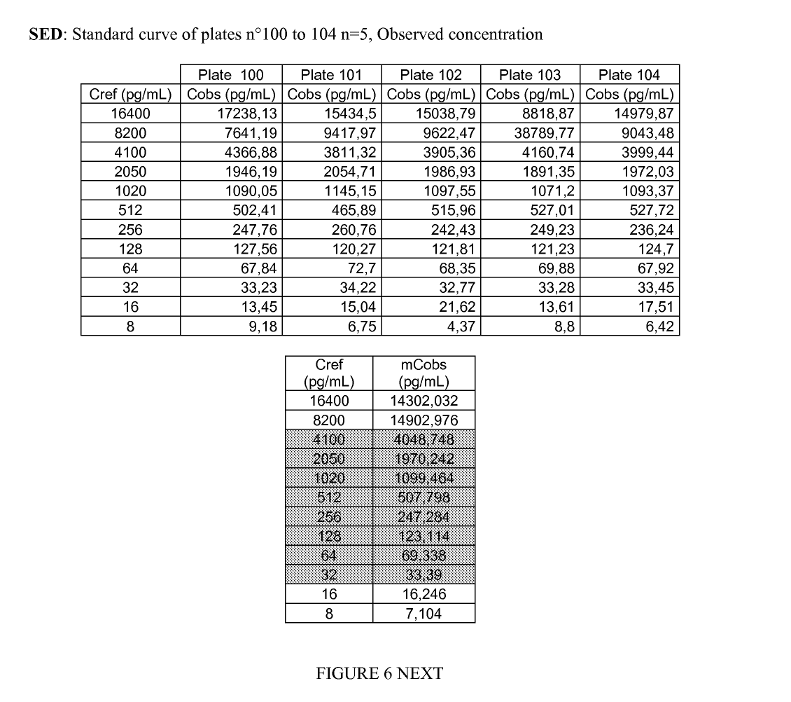

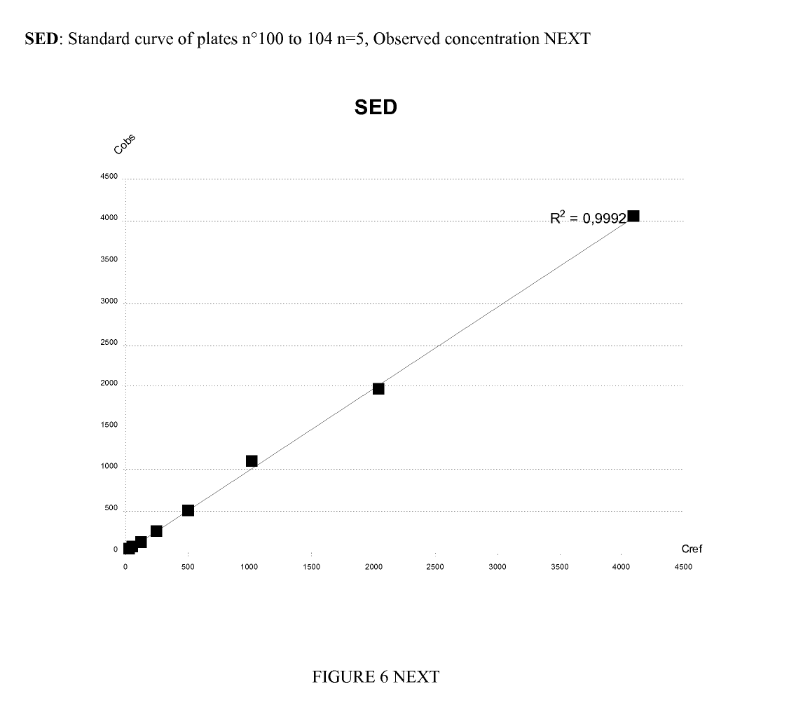

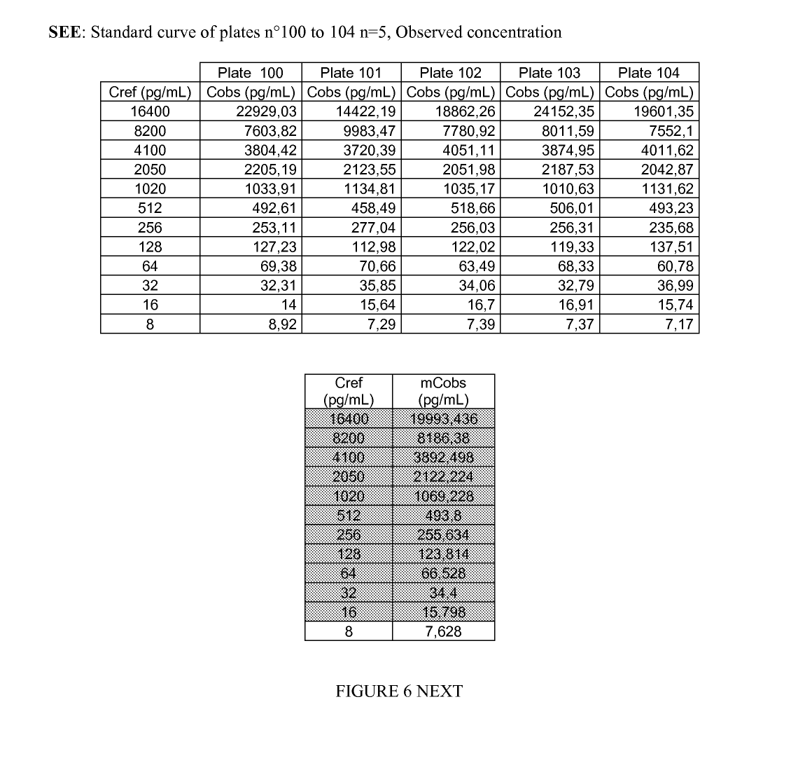

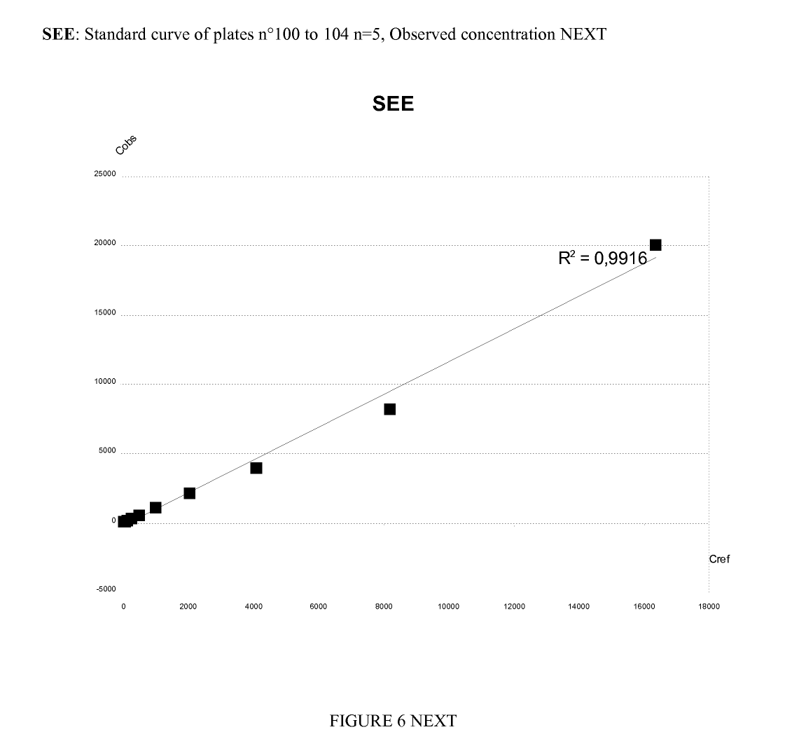

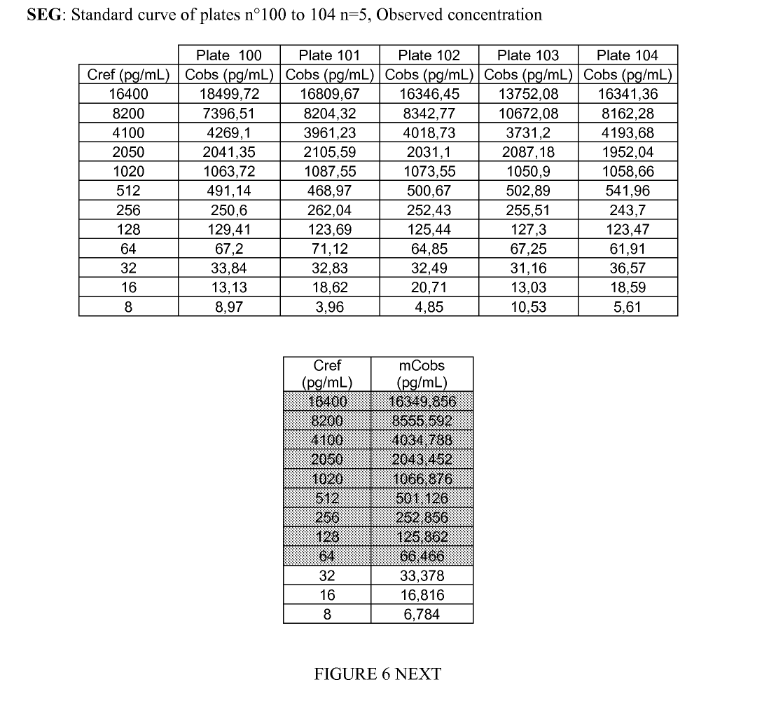

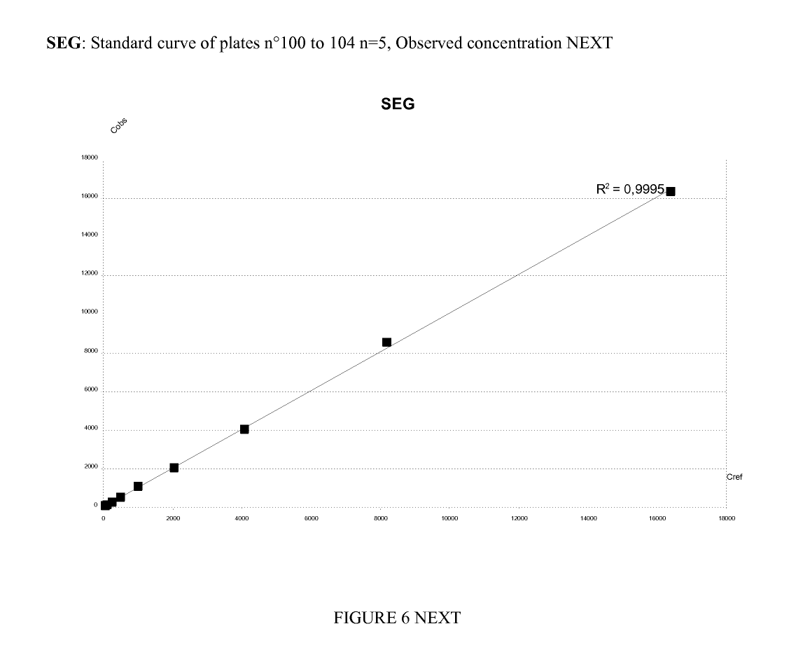

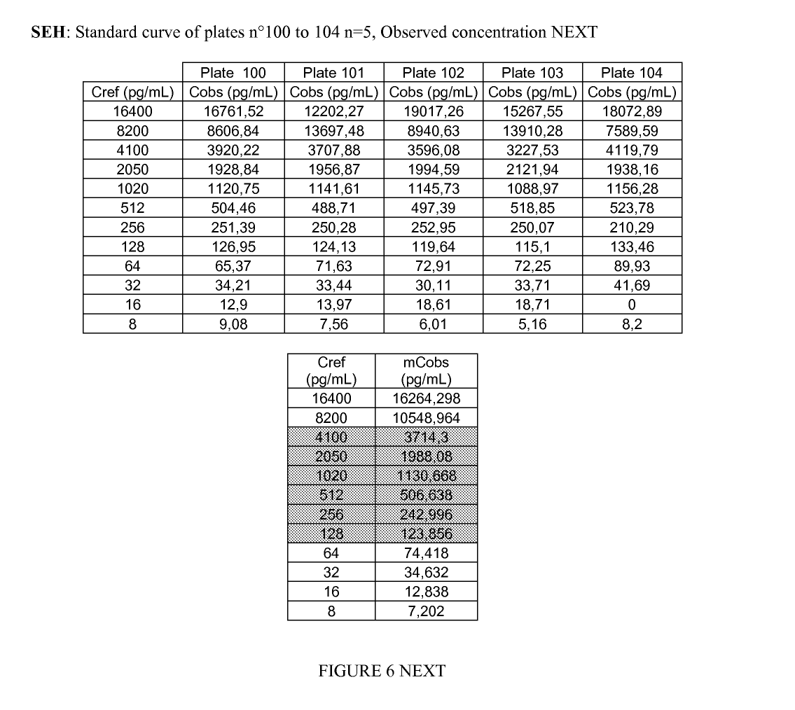

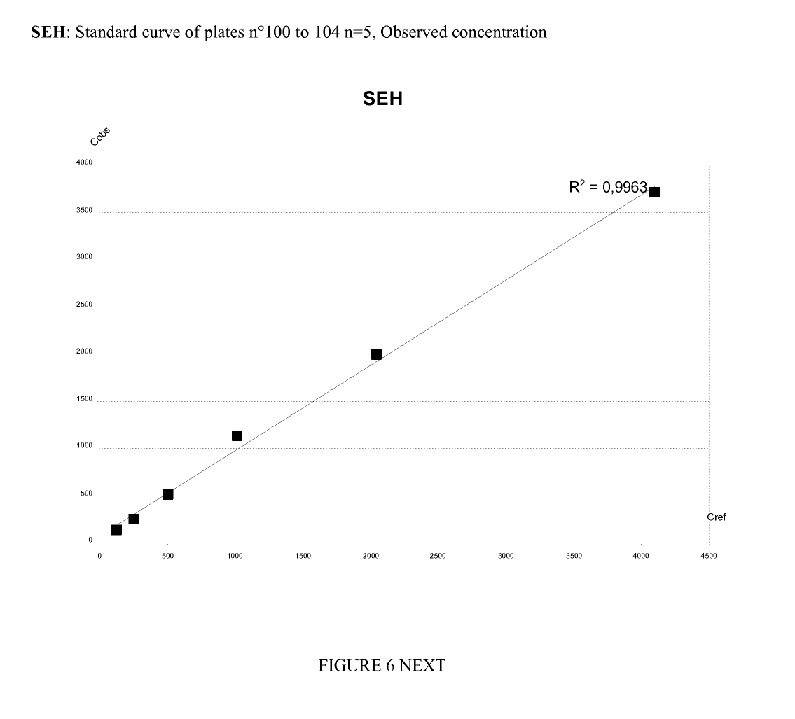

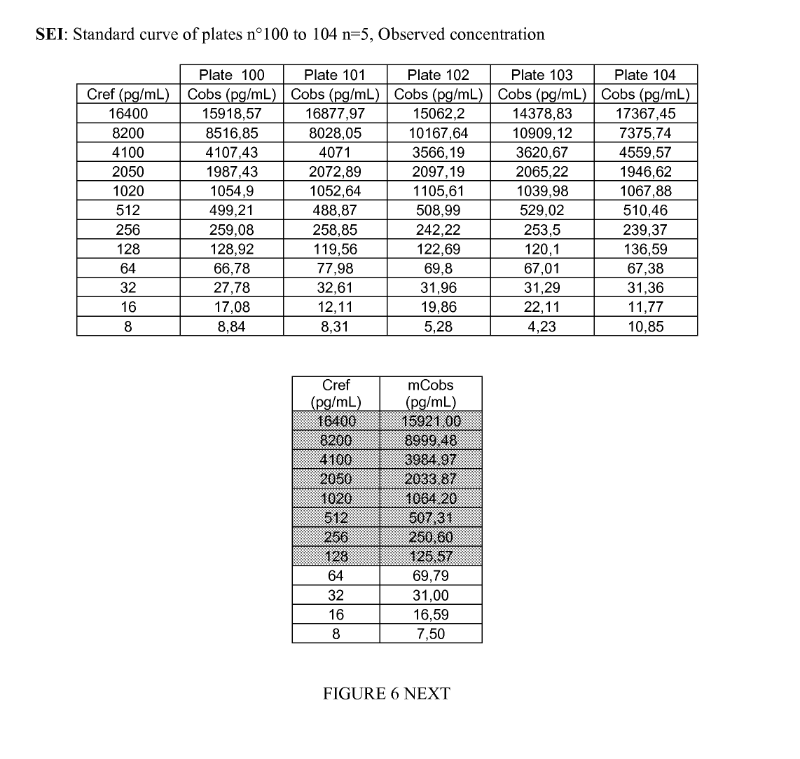

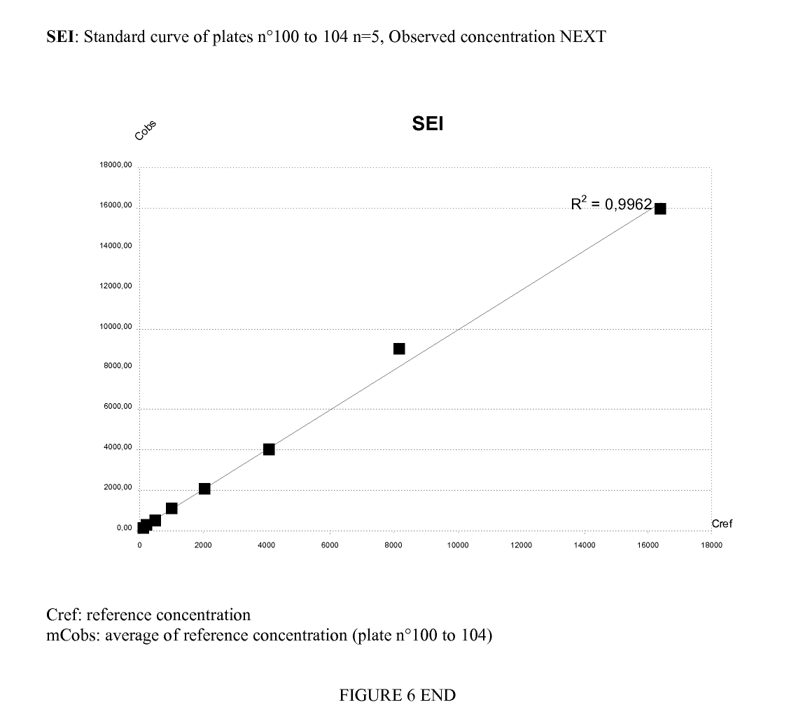

See Linearity of standard in FIG. 6.

TABLE 14

| pg/mL | SEA | SEB | SEC1 | SED | SEE | SEG | SEH | SEI |
|---|---|---|---|---|---|---|---|---|
| R² > 0.99** | 32 to | 8 to | 16 to | 32 to | 16 to | 64 to | 128 to | 128 to |
| | 16400[ | 16400[ | 16400[ | 4100] | 16400[ | 16400[ | 4100[ | 16400[ |

**Whatever PBS buffer or milk considered, strictly comparable values were obtained.

### Example 6

### Titration of the 8 SEs in Different Matrix

### 6.1. Protocol of Matrix Preparation

It was adapted from the method of Macaluso and Lapeyre [Analusis, 28(7): 610-615, 2000] [24], without the PEG 20 000 precipitation, and according to Soejima and al. [2004, fore-mentioned][23] for the concentration by ultrafiltration and delipidation.

Before hand, the 8 SEs were added to the matrix at 4 concentrations (640-320-160-80 pg/mL) likely to be found in contaminated cheese and which the lowest concentration was close to the lower LOQ (particularly for SEH and SEI).
12.5 g of munster (1^{st} price Auchan) were cut in small pieces and added to 25 mL of water in a 50 mL tube (PP) at room-temperature, stirred vigorously 15 sec and grinded by UltraTurrax™ during 1 min. Freeze at −20° C. for several days.
The day of the preparation, homogenates were defrosted and fluidized at 37° C. during several min by shaking. Then the 8 SEs were added at 640-320-160-80 pg/mL in 4 different homogenates, the 5^{th} is the “matrix blank”, stirred vigorously 15 s and left aside for 15 min at room-temperature.

### Caseins Precipitation:

Samples were acidified with HCl (˜5M) until pH 3.5 (check pH with pH indicator sticks pH2 to 9+/−0.5, with Hac solution 0.2M pH3.8 as a positive control). The HCl volume added was written: about 800 μL were necessary. Samples were stirred and centrifuged 10 min at 7000×g, at room-temperature. 20 mL of the supernatant were taken between the pellet and the cream and 50 mM of Tris (1 mL) and 1 mM of CaCl₂ (20 μL) were added then the pH was adjusted at 7.5+/−0.3 with NaOH (˜2M) (pH to check with Lyphan® L669, with HEPES solution 0.5M pH 7.5 as a positive control). The NaOH volume was written: about 300 μL.

The resulting solution was stirred and centrifuged 10 min, room-temperature at 7000×g. ˜15 mL of the supernatant were kept (it may be slightly cloudy).

### Delipidation:

2 mL of HCCI₃ were added to each supernatant, stirred vigorously and centrifuged 10 min at 7000×g at room-temperature and the aqueous phase was kept. A disk of precipitation appeared between the two phases and the supernatant was unclouded.

### Desalting by Ultrafiltration:

4 mL of each supernatant were placed in a ultrafiltration Amicon® 4 (Millipore™) system, with a nominal molecular weight limit of 10 kDa, and centrifugated ˜12 min at 7500 g at room-temperature (rotor 35°). The final volume was lower than 0.8 mL. The retentate was homogenized and the volume was adjusted to 4 mL with TBS (Tris-HCl 40 mM+NaCl 140 mM pH 7.5).

### ×5 Concentration by Ultrafiltration:

Each retentate was re-centrifugated with the same filter unit, then homogenized and the volume was adjusted to 800 μL. That concentrate was more “viscous” than before 5× concentration.

Tween 20 at 0.05% and rabbit aspecific IgG at 10 μg/mL were added to each sample, and frozen at −20° C.

Titration of the 8 SEs were performed the day after according to the general instrumental protocol 4.

### 6.2. Eight SEs Titration in Munster—Results—Conclusion

6.2.1. Recovery Rate in Different Matrix

On the plate 037, there were 10 points (16384 to 32 pg/mL, 2 in 2 of 7SEs(without SED)), prepared (in 50% of whole milk centrifugated and filtered on 0.45 μm or 50% Brain Heart Infusion) in TBS+Tween 20 0.05%+rabbit IgG 100 μg/mL.

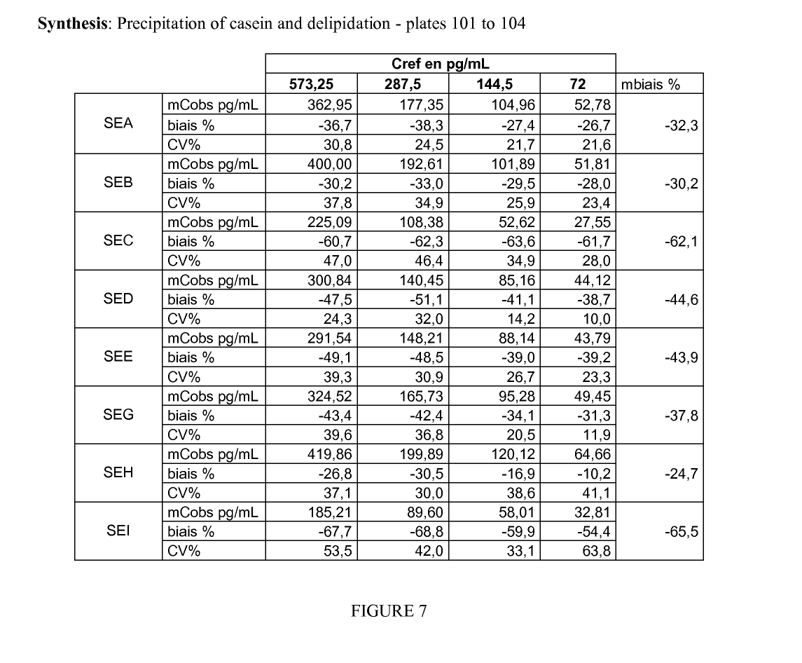

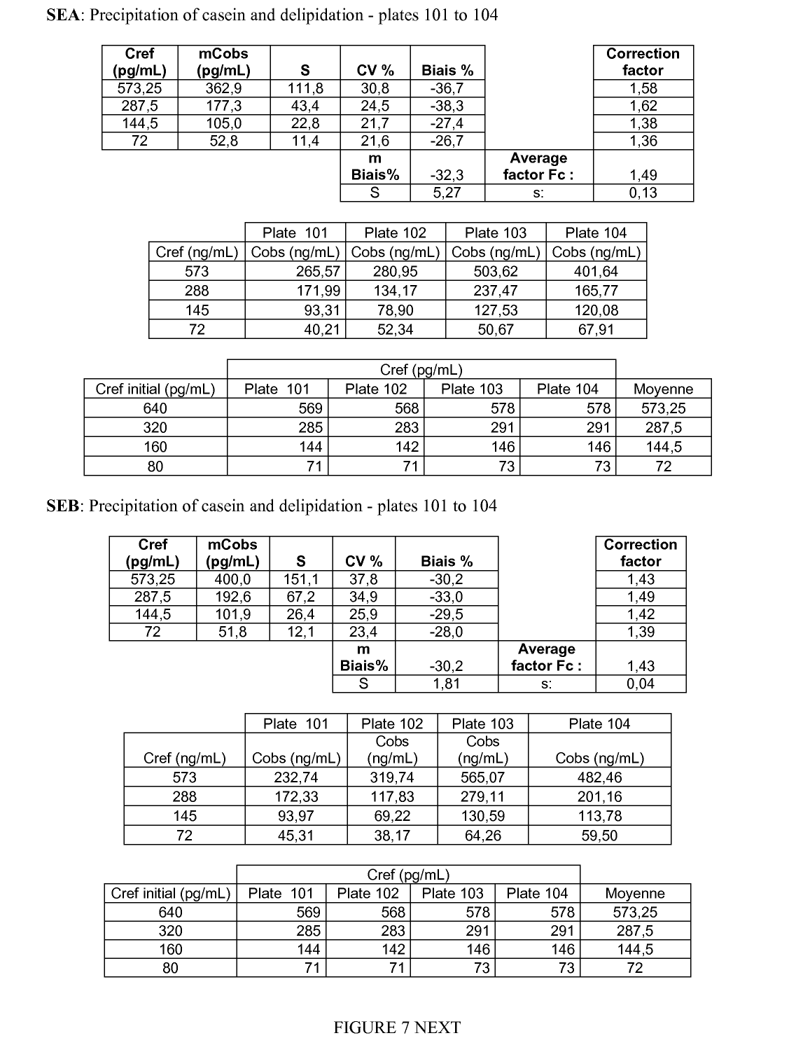

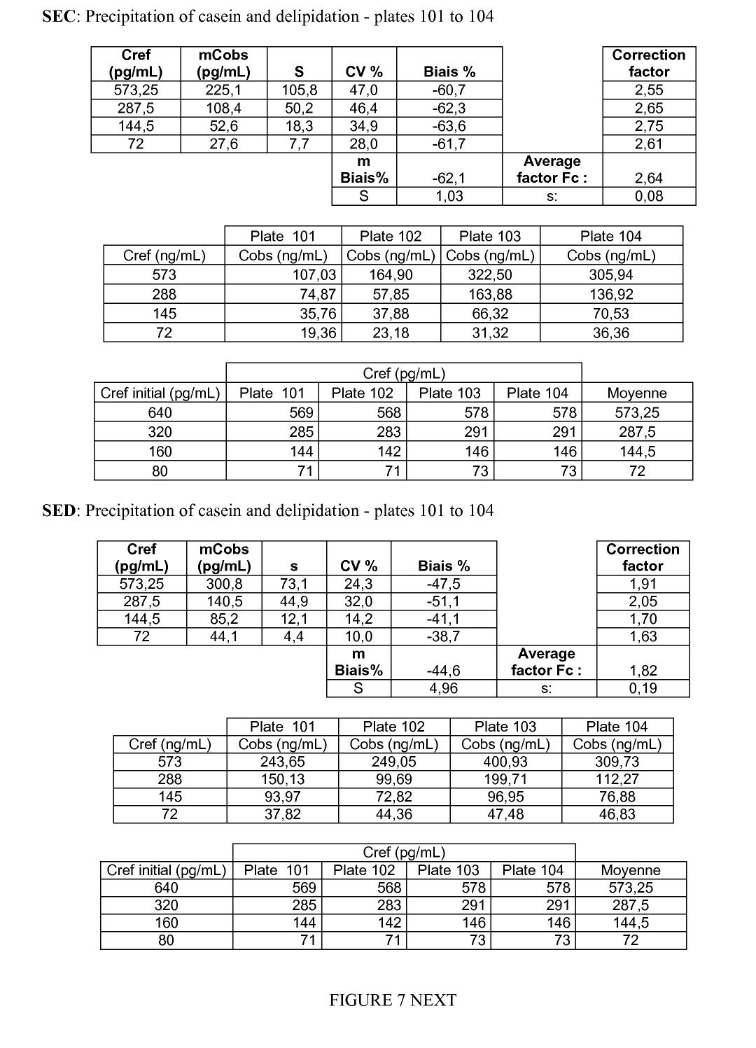

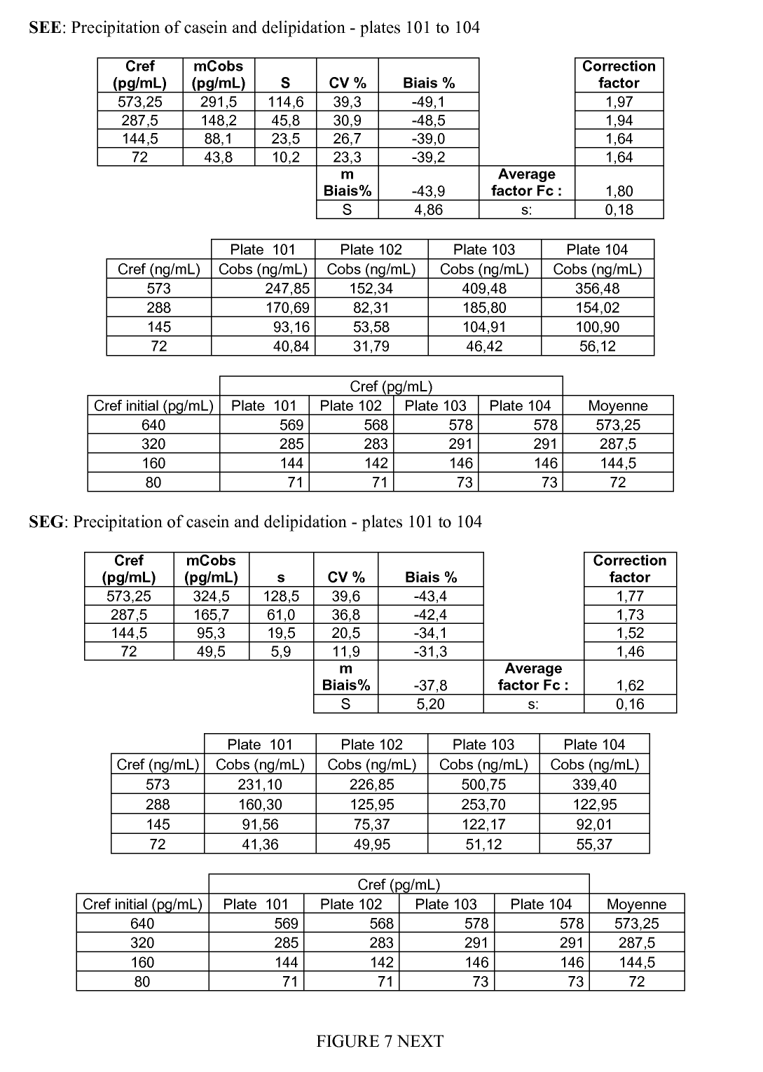

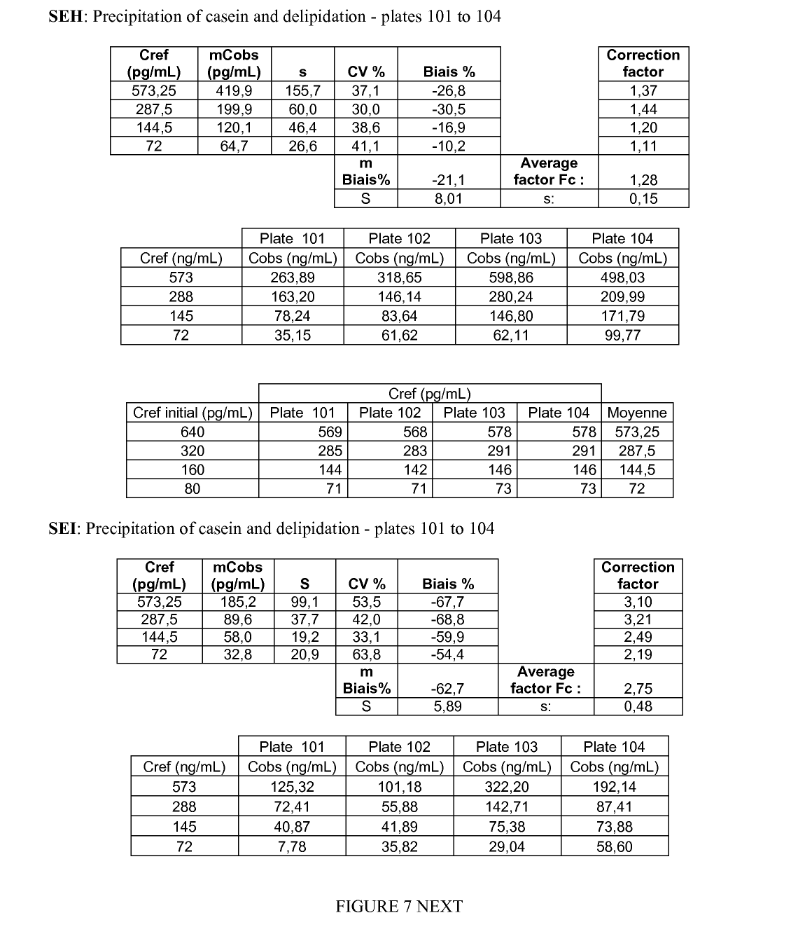

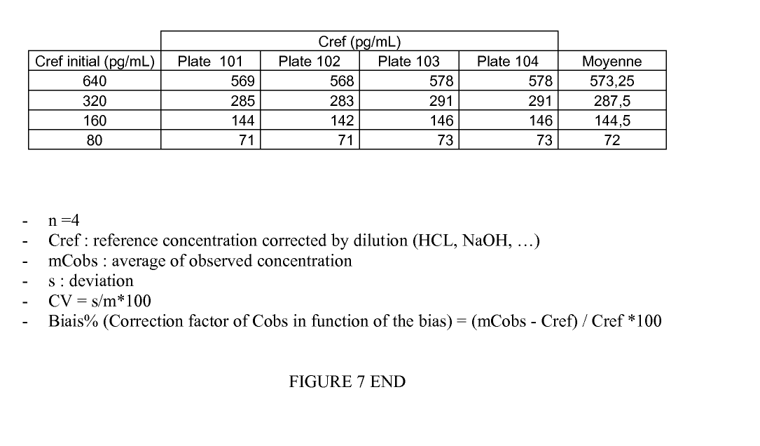

The bias % (Mu HCL) was obtained on Munster after caseins precipitation and delipidation (see also FIG. 7)

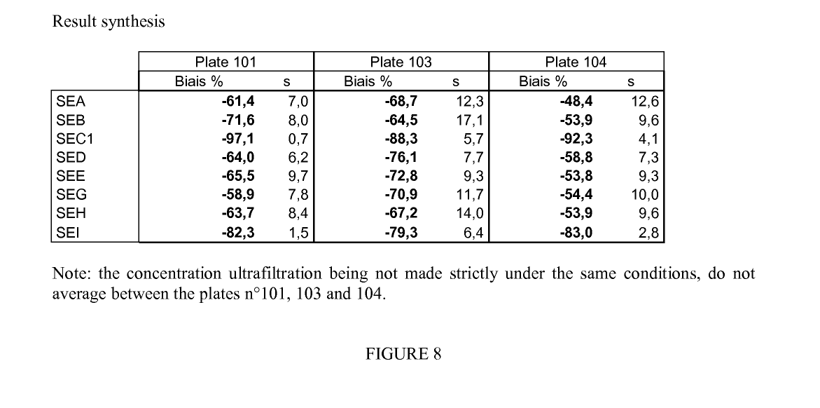

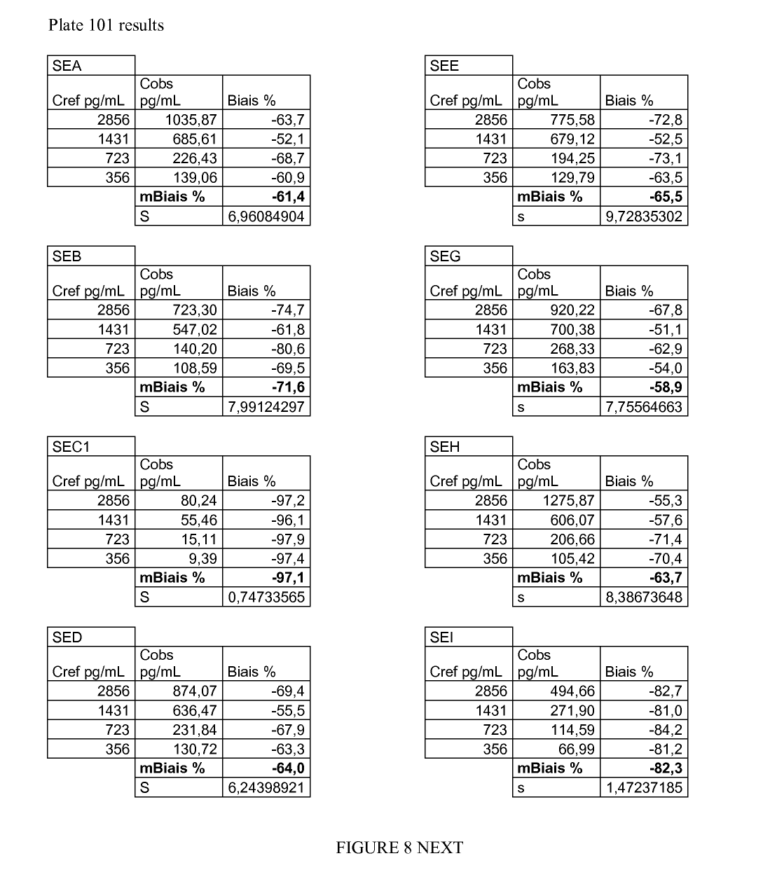

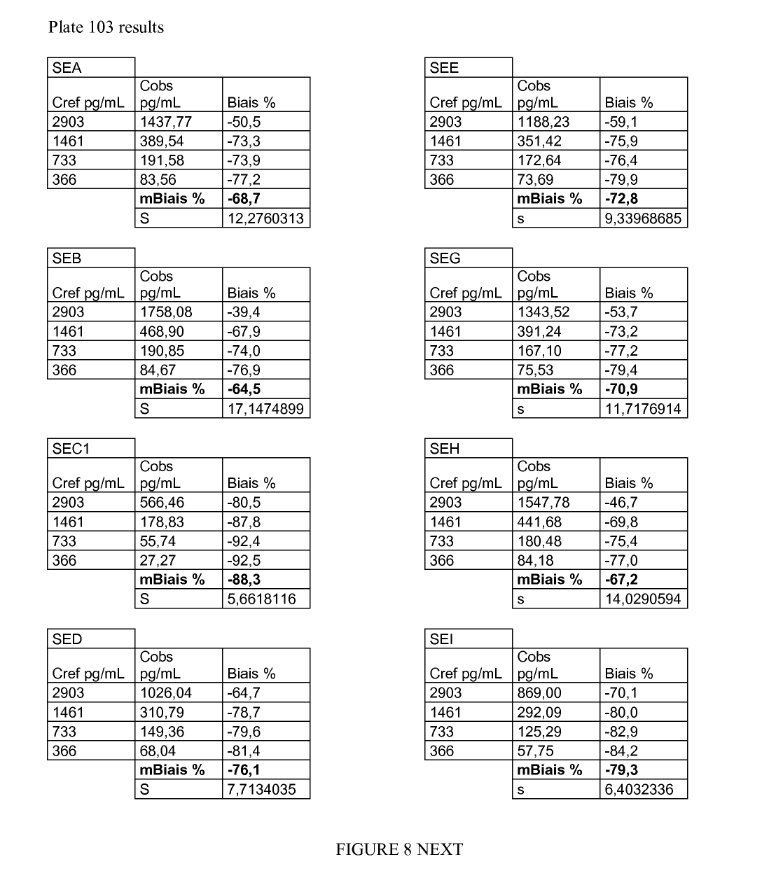

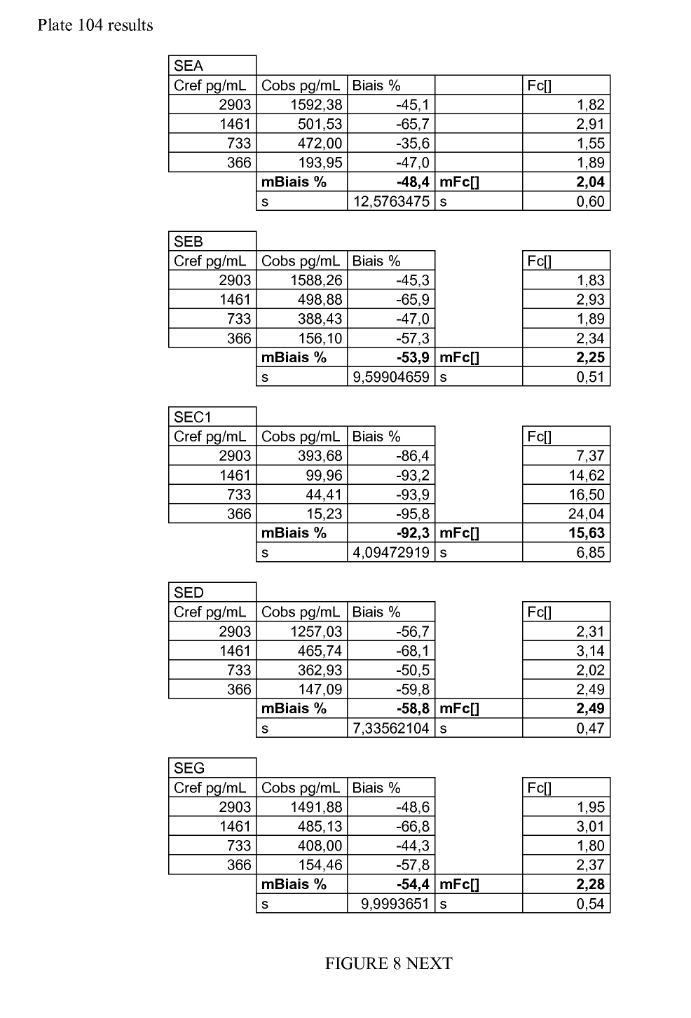

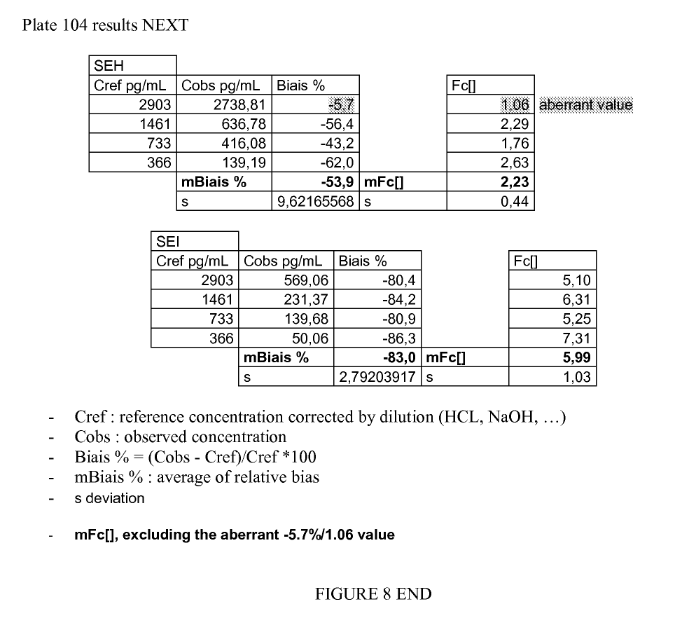

In order to turn the LOQ per g of cheese down, the matrix was concentrated after caseins precipitation and delipidation:
Plate 101: desalting then concentration as exposed in the matrix preparation protocole 6.1 (see also FIG. 8)
Plate 103: ultrafiltration of 10 mL for 1 h at 5000×g at room-temperature with a nominal molecular weight limit of 300 kDa to collect 6 mL of filtrate, of which 4 mLwere desalted and concentrated as exposed in the matrix preparation protocol 6.1. (see also FIG. 8)
Plate 104: ultracentrifugation of 10 mL during 1 h or 3 h at 100 000×g (33300 t/min 50 Ti) then 4 mL were desalted and concentrated as exposed in the matrix preparation protocol 6.1. There were no significant differences between the results of 1 h or 3 h of centrifugation. (see also FIG. 8)

In the table 15 below, the relative bias (%) in relation to TBS+Tween+IgG in standard range was presented:

TABLE 15

| mBias % | SEA | SEB | SEC | SED | SEE | SEG | SEH | SEI |
|---|---|---|---|---|---|---|---|---|
| Lait 50% | −28.5 | −18.0 | −25.9 | ND | −22.5 | −26.1 | −21.2 | −62.6 |
| BHI 50% | −40.9 | −38.8 | −39.2 | ND | −40.1 | −43.3 | −46.1 | −68.0 |
| Mu HCL | −32.3 | −30.2 | −62.1 | −44.6 | −43.9 | −37.8 | −24.7 | −65.5 |
| Mu 101 | −61.4 | −71.6 | −97.1 | −64.0 | −65.5 | −58.9 | −63.7 | −82.3 |
| Mu 103 | −68.7 | −64.5 | −88.3 | −76.1 | −72.8 | −70.9 | −67.2 | −79.3 |
| Mu 104 | −48.4 | −53.9 | −92.3 | −58.8 | −53.8 | −54.4 | −53.9 | −83.0 |

ND: not determined

If average biases were compared, the bias was mostly lower in whole milk than in munster or BHI. SEC showed an important difference between the milk and the munster, and that led to the problem of the protein stability in its secretion environment. SEI had always a high bias, probably due to the insufficient Ab sensibility of that generation.

After concentration, for the 8 SEs absolute value of the biases raise compared with casein precipitation and delipidation: the inhibitory effect of the matrix raised with its concentration, whatever the conditions 101, 103 or 104 which had the less bad result.

6.2.2 Proposal of the Correction of the Loss of 8Ses and Lower LOD in Munster

For all the SEs, an important negative bias has been evaluated, reflecting the inhibitor effect of the matrix. However, that bias could be corrected by a factor defined in the table below: (+/−s (standard deviation)):

After Casein Precipitation and Delipidation

TABLE 16

| CF ± s | SEA | SEB | SEC1 | SED | SEE | SEG | SEH | SEI |
|---|---|---|---|---|---|---|---|---|
| Correction | 1.49 +/− | 1.43 +/− | 2.64 +/− | 1.82 +/− | 1.80 +/− | 1.62 +/− | 1.28 +/− | 2.75 +/− |
| factor | 0.13 | 0.04 | 0.08 | 0.19 | 0.18 | 0.16 | 0.15 | 0.48 |

Note: the concentration Cobs had been introduced regardless the LOQ. Then, due to a negative bias, for SEH and SEI, Cobs are lower than the LOQ for the 72 pg/mL point, which did not have been deleted for that study.

Allowing the dilution factor of 1/3.3 from the matrix preparation and the negative bias correlated to Cf, a new LOQ per g of cheese could be revealed: LOQ pg/g=LOQ pg/mL×3.3×Cf (see the table 17 below).

TABLE 17

| pg/g | SEA | SEB | SEC1 | SED | SEE | SEG | SEH | SEI |
|---|---|---|---|---|---|---|---|---|
| LOQ | 143 | 33 | 96 | 144 | 65 | 230 | 321 | 681 |

After Casein Precipitation Dilapidation and Concentration (Plate 104 Conditions)

TABLE 18

| Cf* +/− s | SEA | SEB | SEC1 | SED | SEE | SEG | SEH | SEI |
|---|---|---|---|---|---|---|---|---|
| Correction | 2.04 +/− | 2.25 +/− | 15.63 +/− | 2.49 +/− | 2.24 +/− | 2.28 +/− | 2.23 +/− | 5.99 +/− |
| Factor* | 0.60 | 0.51 | 6.85 | 0.47 | 0.53 | 0.54 | 0.44 | 1.03 |

Allowing the concentration factor of 5/3.3 resulting from the matrix preparation and the negative bias reflected by Cf*, a new lower LOQ per g of cheese could be revealed: Lower LOQ pg/g of cheese=lower LOQ pg/mL×3.3/5×Cf (see the table 19 below).

TABLE 19

| pg/g | SEA | SEB | SEC1 | SED | SEE | SEG | SEH | SEI |
|---|---|---|---|---|---|---|---|---|
| Lower LOQ | 39 | 10 | 113 | 39 | 18 | 65 | 112 | 297 |

Overall, the lower LOQ per g of cheese drop after concentration, apart from SEC1. But since Cf*>Cf, the result uncertainty raises.

The depleted polyclonal antibodies developed here could be used in different tests, among which the Luminex™ technology. These antibodies conserved a high affinity and gathered the possibility to detect all SEs already designed to be responsible for collective food-born intoxications.

### Example 7

### Titration of SEA and SED Produced by S. aureus in Cheese

This assay was carried out to test whether enterotoxins produced by different Staphylococcus aureus isolates could be detected in cheeses: Munster or Babybel®.

### 7.1. Strain 361F

This strain produces SEA and SED often implicated in foodborne disease. Bacteria were added on the first day (D0) of cheese making at four different concentrations: 10³, 10⁴, 10⁵, 10⁶ colonies forming units per milliliter. Samples were collected at different days:

D1 and D21 for Babybel®

D1 and D15 for Munster. At D15, the heart (H) of the cheese and crust (C) were separated into two separate samples.

The collected samples were extracted using the protocol described in 6.1 and titrated using the protocol described in 4. After correction for the extraction volume, the results were expressed as pg enterotoxin/g of cheese (n=3).

### Munster

| | | | |
|---|---|---|---|
| 10³ CFU/mL | 10⁴ CFU/mL | 10⁵ CFU/mL | 10⁶ CFU/mL |

D1

| | | | | |
|---|---|---|---|---|
| SEA | 7 ± 0.6 | 84 ± 6.1 | 787 ± 9.7 | 2582 ± 82.6 |
| SED | ND | 26 ± 3.5 | 183 ± 7.0 | 532 ± 16.2 |

D15H

| | | | | |
|---|---|---|---|---|
| SEA | 6 ± 2.0 | 75 ± 6.4 | 588 ± 52.3 | 2086 ± 146.3 |
| SED | ND | 14 ± 1.1 | 123 ± 15.1 | 364 ± 62.7 |

D15C

| | | | | |
|---|---|---|---|---|
| SEA | 9 ± 1.3 | 159 ± 10.6 | 1023 ± 132.2 | 2590 ± 367.0 |
| SED | ND | 89 ± 26.3 | 190 ± 60.2 | 415 ± 127.0 |

ND: not detected

### Babybel®

| | | | |
|---|---|---|---|
| 10³ CFU/mL | 10⁴ CFU/mL | 10⁵ CFU/mL | 10⁶ CFU/mL |

D1

| | | | | |
|---|---|---|---|---|
| SEA | 105 ± 4.6 | 706 ± 60.2 | 1802 ± 5.0 | 2529 ± 27.5 |
| SED | 30 ± 4.6 | 230 ± 16.6 | 471 ± 11.6 | 640 ± 69.6 |

D21

| | | | | |
|---|---|---|---|---|
| SEA | 249 ± 26.5 | 996 ± 33.6 | 4535 ± 653.6 | 7768 ± 1405 |
| SED | 65 ± 18.1 | 226 ± 16.0 | 917 ± 237.2 | 1364 ± 217.1 |

This assay detected enterotoxins SEA and SED produced by S. aureus in cheeses from densities greater than 10³ CFU/mL. The final titer increased with the bacterial count, the length of the maturing period and the two enterotoxins rather accumulated in the crust of Munster.

### 7.2. Other Strains

In the same conditions the production of other Enterotoxins (pg/g) in cheese (Munster and Babybel®) contaminated with S. aureus was tested but only at 10⁴ CFU/mL (n=3).

### Strain A79: SEA+ and SEB+

| Munster | D1 | D15H | D15C |
|---|---|---|---|
| SEA | 72 ± 14.2 | 68 ± 10.3 | 73 ± 13.0 |
| SEB | 23 ± 2.0 | 14 ± 1.3 | 26 ± 7.1 |

| Babybel ® | D1 | D21 |
|---|---|---|
| SEA | 945 ± 132.6 | 1893 ± 178.4 |
| SEB | 444 ± 31.0 | 756 ± 179.7 |

### Strain P4: SEC+, SEG+, SEH+ and SEI+

| Munster | D1 | D15H | D15C |
|---|---|---|---|
| SEC | ND | ND | ND |
| SEG | ND | ND | ND |
| SEH | ND | ND | ND |
| SEI | ND | ND | ND |

ND: not detected

| Babybel ® | D1 | D21 |
|---|---|---|
| SEC | ND | ND |
| SEG | ND | ND |
| SEH | 69 ± 22.5 | 416 ± 8.9 |
| SEI | ND | ND |

ND: not detected

### Strain A67: SEE+

| Munster | D1 | D15H | D15C |
|---|---|---|---|
| SEE | 531 ± 10.3 | 634 ± 20.1 | 1888 ± 285.2 |

| Babybel ® | D1 | D21 |
|---|---|---|
| SEE | 1967 ± 78.0 | 6307 ± 611.4 |

### Strain P6: SEH+

| Munster | D1 | D15H | D15C |
|---|---|---|---|
| SEH | ND | ND | 512 ± 280.3 |

| Babybel ® | D1 | D21 |
|---|---|---|
| SEH | 130 ± 77.2 | 592 ± 102.8 |

ND: not detected

The search for the production of enterotoxins in cheese from the five strains above showed that this production was dependent on the strain. If the test detected SEA, SEB, SED, SEE and SEH, it did not reveal clearly the production of SEC, SEG and SEI.

### Example 8

### Titration of the 8 SES in Meat and Sauce

Four foods were selected based on animal proteins involved in the foodborne diseases: duck mousse (DM), liver mousse of pork (PM), custard sauce (CS) and pepper sauce (PS).

### 8.1. Protocol of Matrix Preparation

This protocol was adapted from that used for the cheese described in 6.1 with modifications:
8 SEs were added to the matrix at 800 pg/mL and a blank was prepared for each food.
Precipitation of casein is performed only for pepper sauce.
In all cases, after centrifugation 10 min at 7000×g, the cloudy supernatants were non-filterable later during the washing steps of the ELISA. Also, lipids were extracted with two volumes of chloroform. After centrifugation, a disk of precipitation appeared between the two phases and the supernatants were unclouded and ready for desalting and concentration by ultrafiltration.

### 8.2. Eight SES Titration—Results—Conclusion

Bias (%) after desalting

| % | SEA | SEB | SEC | SED | SEE | SEG | SEH | SEI |
|---|---|---|---|---|---|---|---|---|
| DM | +36 | +40 | +45 | +33 | +1 | +28 | +36 | −56 |
| PM | +7 | +30 | +3 | +3 | −11 | +5 | +5 | −63 |
| CS | +11 | +21 | −6 | +11 | −16 | +5 | +12 | −81 |
| PS | −21 | −37 | −21 | −47 | −43 | −38 | −16 | −82 |

Bias (%) after concentration (×5)

| % | SEA | SEB | SEC | SED | SEE | SEG | SEH | SEI |
|---|---|---|---|---|---|---|---|---|
| DM | +8 | +9 | −7 | +3 | −30 | −3 | +16 | −73 |
| PM | −25 | −35 | −25 | −36 | −43 | −34 | −6 | −80 |
| CS | −13 | −18 | −20 | −12 | −32 | −19 | −22 | −85 |
| PS | −51 | −70 | −60 | −80 | −71 | −59 | −53 | −93 |

The bias after desalting was lower than after concentration.

Remark: generally, samples were more viscous after concentration, especially pepper sauce.

For several samples the bias was positive: duck mousse for example, this bias could come from a poor dissemination of toxins at the step of contamination. SEI had always a high bias, probably due to the insufficient Ab sensibility of that generation.

The depleted polyclonal antibodies developed here could be used in different tests, among which the Luminex™ technology. These antibodies conserved specificity and affinity and authorize the possibility to detect all SEs already designed to be responsible for collective food-born intoxications.

### LIST OF REFERENCES

1. Jones and Kahn, J. Bacteriol., 166: 29-33, 1986
2. Betley and Mekalanos, J. Bacteriol., 170(1): 34-41, 1988
3. Couch and al., J. Bacteriol., 170: 2954-2960, 1988
4. Bayles and landolo, J. Bacteriol., 171: 4799-4806, 1989
5. Dingues and al., Clin. Microbiol. Rev., 13: 16-34, 2000
6. Bergdoll and al., J. Bacteriol., 90(5): 1481-1485, 1965
7. Marr and al., Infect. Immun., 61: 4254-4262, 1993
8. Bergdoll, Lancet, 1: 1017-1021, 1981
9. Blomster-Hautamaa and al., J. Biol. Chem., 261: 15783-15786, 1986
10. Lina and al., J. Infect. Dis., 189(12): 2334-2336, 2004
11. Ren et al., J. Exp. Med., 180(5): 1675-1683, 1994
12. Jarraud and al., J. Immunol., 166(1): 669-677, 2001
13. Orwin and al., Infect. Immun., 69(1): 360-366, 2001
14. Letertre and al., Mol. Cell Probes, 17: 227-235, 2003
15. Omoe and al., J. Clin. Microbiol., 40: 857-862, 2002
16. Su and Wong, J. Food Prot., 59(3): 327-330, 1996
17. Munson and al., Infect. Immun., 66: 3337-3348, 1998
18. Ono and al., Infect. Immun., 76(11): 4999-5005, 2008
19. Smith and Johnson, Gene, 67(1): 31-40, 1988
20. Habig and al., J. Biol. Chem., 249: 7130-7139, 1974
21. Gampfer et al., Vaccine, 20: 3675-3684, 2002
22. Borkowski and al., Clin. Tech. Small Anim. Pract., 14.(1): 44-49, 1999
23. Soejima and al., Int. J. Food Microbiol., 93: 185-194, 2004
24. Macaluso et al., Analusis, 28(7): 610-615, 2000

1. Immunological composition comprising a mixture of at least two depleted polyclonal antibodies each depleted polyclonal antibody being raised against one staphylococcal enterotoxin, and a pharmaceutically acceptable carrier.

2. Immunological composition according to claim 1, comprising a mixture of at least eight depleted polyclonal antibodies each depleted polyclonal antibody being raised against one specific staphylococcal enterotoxin, and a pharmaceutically acceptable carrier.

3. Immunological composition according to claim 1, wherein each depleted polyclonal antibody is raised against one staphylococcal enterotoxin chosen from the group consisting of the staphylococcal enterotoxins A, B, C, D, E, G, H, and I.

4. Method for multiplex detection of staphylococcal enterotoxins, comprising:
a) contacting a sample with an immunological composition as defined in claim;
b) detecting potential immunological complexes formed.

5. Use of an immunological composition as defined in claim 1, as a diagnostic tool of a staphylococcal enterotoxin contamination.

6. Kit for the multiplex detection of staphylococcal enterotoxins, comprising an immunological composition as defined in claim 1, and means for the detection of immunological complexes.

7. Method for the preparation of a depleted polyclonal antibody raised against a staphylococcal enterotoxin, comprising:
a) providing an anti-enterotoxin polyclonal antibody previously obtained from the immunization of a non-human animal with a staphylococcal enterotoxin;
b) purification of said anti-enterotoxin polyclonal antibody using at least two successive depletion steps against immunization-unrelated staphylococcal enterotoxins and which order is chosen to abolish cross-reactions with said immunization-unrelated staphylococcal enterotoxins from the strongest cross-reaction to the weaker cross-reaction.

8. Method according to claim 7 for the preparation of a depleted anti-enterotoxin A polyclonal antibody, comprising:
a) providing an anti-enterotoxin A polyclonal antibody previously obtained from the immunization of a non-human animal with the staphylococcal enterotoxin A;
b) purification of said anti-enterotoxin A polyclonal antibody following 5 successive depletion steps against the immunization-unrelated staphylococcal enterotoxin E, E, I, B, then D.

9. Method according to claim 7 for the preparation of a depleted anti-enterotoxin B polyclonal antibody, comprising:
a) providing an anti-enterotoxin B polyclonal antibody previously obtained from the immunization of a non-human animal with the staphylococcal enterotoxin B;
b) purification of said anti-enterotoxin B polyclonal antibody following 3 successive depletion steps against the immunization-unrelated staphylococcal enterotoxin C1, C1, then G.

10. Method according to claim 7 for the preparation of a depleted anti-enterotoxin C polyclonal antibody, comprising:
a) providing an anti-enterotoxin C polyclonal antibody previously obtained from the immunization of a non-human animal with the staphylococcal enterotoxin C;
b) purification of said anti-enterotoxin C1 polyclonal antibody following 3 successive depletion steps against immunization-unrelated staphylococcal enterotoxin B, B, then G;

11. Method according to claim 7 for the preparation of a depleted anti-enterotoxin D polyclonal antibody, comprising:
a) providing an anti-enterotoxin D polyclonal antibody previously obtained from the immunization of a non-human animal with the staphylococcal enterotoxin D;
b) purification of said anti-enterotoxin D polyclonal antibody following 3 successive depletion steps against the immunization-unrelated staphylococcal enterotoxin A, A, then E.

12. Method according to claim 7 for the preparation of a depleted anti-enterotoxin E polyclonal antibody, comprising:
a) providing an anti-enterotoxin E polyclonal antibody previously obtained from the immunization of a non-human animal with the staphylococcal enterotoxin E;
b) purification of said anti-enterotoxin E polyclonal antibody following 4 successive depletion steps against the immunization-unrelated staphylococcal enterotoxin A, A, I, then C1.

13. Method according to claim 7 for the preparation of a depleted anti-enterotoxin G polyclonal antibody, comprising:
a) providing an anti-enterotoxin G polyclonal antibody previously obtained from the immunization of a non-human animal with the staphylococcal enterotoxin G;
b) purification of said anti-enterotoxin A polyclonal antibody following 4 successive depletion steps against the immunization-unrelated staphylococcal enterotoxin A, B, I, then C1.

14. Method according to claim 7 for the preparation of a depleted anti-enterotoxin H polyclonal antibody, comprising:
a) providing an anti-enterotoxin H polyclonal antibody previously obtained from the immunization of a non-human animal with the staphylococcal enterotoxin H;
b) purification of said anti-enterotoxin A polyclonal antibody following 2 successive depletion steps against the immunization-unrelated staphylococcal enterotoxin B, then D.

15. Method according to claim 7 for the preparation of a depleted anti-enterotoxin I polyclonal antibody, comprising:
a) providing an anti-enterotoxin I polyclonal antibody previously obtained from the immunization of a non-human animal with the staphylococcal enterotoxin I;
b) purification of said anti-enterotoxin I polyclonal antibody following 5 successive depletion steps against immunization-unrelated staphylococcal enterotoxin E, C1, G, B, then A.


SEQUENCE LISTING (22 sequences)

SEQ ID NO: 1 - DNA, 37 bp, Artificial Sequence
  Primer BamH1-setBf
agtaaggatccgagagagtcaaccagatcctaaacca

SEQ ID NO: 2 - DNA, 42 bp, Artificial Sequence
  Primer EcoR1-setBr
aagtatagaattcactaacacttcatatttactttctaaaat

SEQ ID NO: 3 - DNA, 39 bp, Artificial Sequence
  primer EcoR1-setC1f
aagtatagaattcgagagccaaccagaccctacgccaga

SEQ ID NO: 4 - DNA, 39 bp, Artificial Sequence
  Primer EcoR1-setC1r
aagtatagaattctactttcactttttatatcaaaatgg

SEQ ID NO: 5 - DNA, 42 bp, Artificial Sequence
  Primer EcoR1-setDf
aagtatagaattcaaacattcttatgcagataaaaatccaat

SEQ ID NO: 6 - DNA, 39 bp, Artificial Sequence
  Primer EcoR1-setDr
aagtatagaattcttgaaatggctttagtgtctgatgtt

SEQ ID NO: 7 - DNA, 37 bp, Artificial Sequence
  Primer EcoR1-setEf
agtaaggatccagcgaagaaataaatgaaaaagattt

SEQ ID NO: 8 - DNA, 39 bp, Artificial Sequence
  Primer EcoR1-setEr
aagtatagaattcagttgtgtataaatacaaatcaatat

SEQ ID NO: 9 - DNA, 36 bp, Artificial Sequence
  Primer EcoR1-seGf
agtaaggatcccaacccgatcctaaattagacgaac

SEQ ID NO: 10 - DNA, 42 bp, Artificial Sequence
  Primer EcoR1-SeGr
aagtatgaattctcagtgagtattaagaaatacttccatttt

SEQ ID NO: 11 - DNA, 37 bp, Artificial Sequence
  Primer EcoR1-setHf
agtaaggatccgaagatttacacgataaaagtgagtt

SEQ ID NO: 12 - DNA, 39 bp, Artificial Sequence
  Primer EcoR1-setHr
aagtatagaattctgatctagaaattttcattgattata

SEQ ID NO: 13 - DNA, 37 bp, Artificial Sequence
  Primer BamH1-setIf
agtaaggatcccaaggtgatattggtgtaggtaactt

SEQ ID NO: 14 - DNA, 38 bp, Artificial Sequence
  Primer EcoR1-setIr
aagtatagaattcgttactatctacatatgatatttcg

SEQ ID NO: 15 - protein, 238 aa, Staphylococcus aureus
GPLGSSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 16 - protein, 244 aa, Staphylococcus aureus
GPLGSESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 17 - protein, 247 aa, Staphylococcus aureus
GPLGSPEFESQPDPTPDELHKASKFTGLMENMKVLYDDHYVSATKVKSVDKFLAHDLIYNISDKKLKNYDKVKTELLNEGLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNG

SEQ ID NO: 18 - protein, 207 aa, Staphylococcus aureus
KHSYADKNPIIGENKSTGDQFLENTLLYKKFFTDLINFEDLLINFNSKEMAQHFKSKNVDVYPIRYSINCYGGEIDRTACTYGGVTPHEGNKLKERKKIPINLWINGVQKEVSLDKVQTDKKNVTVQELDAQARRYLQKDLKLYNNDTLGGKIQRGKIEFDSSDGSKVSYDLFDVKGDFPEKQLRIYSDNKTLSTEHLHIDIYLYEK

SEQ ID NO: 19 - protein, 235 aa, Staphylococcus aureus
GPLGSSEEINEKDLRKKSELQRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 20 - protein, 241 aa, Staphylococcus aureus
GPLGSPEFQPDPKLDELNKVSDYKNNKGTMGNVMNLYTSPPVEGRGVINSRQFLSHDLIFPIEYKSYNEVKTELENTELANNYKDKKVDIFGVPYFYTCIIPKSEPDINQNFGGCCMYGGLTFNSSENERDKLITVQVTIDNRQSLGFTITTNKNMVTIQELDYKARHWLTKEKKLYEFDGSAFESGYIKFTEKNNTSFWFDLFPKKELVPFVPYKFLNIYGDNKVVDSKSIKMEVFLNTH

SEQ ID NO: 21 - protein, 222 aa, Staphylococcus aureus
GPLGSEDLHDKSELTDLALANAYGQYNHPFIKENIKSDEISGEKDLIFRNQGDSGNDLRVKFATADLAQKFKNKNVDIYGASFYYKCEKISENISECLYGGTTLNSEKLAQERVIGANVWVDGIQKETELIRTNKKNVTLQELDIKIRKILSDKYKIYYKDSEISKGLIEFDMKTPRDYSFDIYDLKGENDYEIDKIYEDNKTLKSDDISHIDVNLYTKKKV

SEQ ID NO: 22 - protein, 223 aa, Staphylococcus aureus
GPLGSQGDIGVGNLRNFYTKHDYIDLKGVTDKNLPIANQLEFSTGTNDLISESNNWDEISKFKGKKLDIFGIDYNGPCKSKYMYGGATLSGQYLNSARKIPINLWVNGKHKTISTDKIATNKKLVTAQEIDVKLRRYLQEEYNIYGHNNTGKGKEYGYKSKFYSGFNNGKVLFHLNNEKSFSYDLFYTGDGLPVSFLKIYEDNKIIESEKFHLDVEISYVDSN